(19) United States
(12) Patent Application Publication
(10) Pub. No.: US 2023/0400416 A1
(43) Pub. Date: Dec. 14, 2023

(54) AUTOMATIC DETERMINATION SYSTEM, AUTOMATIC DETERMINATION METHOD, AND COMPUTER-READABLE RECORDING MEDIUM HAVING STORED THEREIN AUTOMATIC DETERMINATION PROGRAM

(71) Applicant: KANEKA CORPORATION, Osaka (JP)

(72) Inventors: Shigehiko MIYAMOTO, Hyogo (JP); Sotaro SANO, Hyogo (JP)

(21) Appl. No.: 18/248,756

(22) PCT Filed: Oct. 12, 2021
(86) PCT No.: PCT/JP2021/037779

§ 371 Date: Apr. 12, 2023

(30) Foreign Application Priority Data
Oct. 16, 2020 (JP) 2020-174616

(51) Int. Cl. G01N 21/78; G16H 10/40; G16H 30/20

(52) U.S. Cl. CPC G01N 21/78; G16H 10/40; G16H 30/20

(57) ABSTRACT

Provided is, for example, an automatic determination system. The automatic determination system includes an information storage unit configured to store result-of-detection image information acquired from a chromatographic strip support tool carrying the result-of-detection image information, the result-of-detection image information representing transcribed information regarding expression information obtained on a chromatographic strip that has been through testing, and a result-of-determination deriving unit configured to check an image included in the result-of-detection image information against a data table, and derive a result of determination included in the data table and corresponding to the image.

### TECHNICAL FIELD

The present invention relates to an automatic determination system, an automatic determination method, and an automatic determination program.

### BACKGROUND ART

Hitherto, for example, immunochromatography and nucleic acid chromatography have been known as the methods for analyzing biological components such as proteins and nucleic acids contained in liquid analytes. Test strips to which, for example, the principle of paper chromatography is applied (these test strips may hereinafter be referred to as chromatographic strips) are used in these analyzing methods.

The chromatographic strip includes, for example, an analyte supply section to which an analyte is supplied, a membrane section in which the analyte is developed, and an absorption pad section configured to absorb the analyte. After the analyte is supplied to the analyte supply section and developed with a developing solution, a line coloring (color development on a test line) occurs in the membrane section when a predetermined biological component is present. By reading this line coloring, it is possible to detect that the supplied analyte contains the biological component.

To read the line coloring in the membrane section, one typically sets the chromatographic strip on a determination assisting tool (hereinafter, may be referred to as “chromatographic strip support tool”), on which detection items corresponding to the locations of line colorings are written, and visually checks the detection item at which a line coloring is seen.

With a view to, for example, performing quantitative analyses based on the intensity (density) of a line coloring and minimizing human errors of mistaken reading that may occur when there are a plurality of colored test lines, studies are being made into techniques for automating reading of a line coloring on a chromatographic strip by use of a device including an image capturing unit such as an image sensor, instead of visual checking by humans. For example, a technique proposed as such a technique captures an image of a developed color image (or of a color-developing portion) during chromatography using an image sensor, and applies various image processes to the captured image, to thereby perform a chromatographic quantitative measurement (for example, see PTLs 1 and 2).

### CITATION LIST

### Patent Literature

PTL 1: Japanese Patent Application Laid-Open (JP-A) No. 2000-266752
PTL 2: JP-A No. 2009-98080

### SUMMARY OF INVENTION

### Technical Problem

The intensity (density) of a line coloring on a chromatographic strip may be sometimes weak and insufficient, because it is dependent on the abundance or concentration of the target substance in the supplied analyte. When a line coloring on a chromatographic strip is weak as mentioned, automated reading by use of a device including, for example, an image sensor may fail to correctly read the line coloring (i.e., mistaken determination may occur due to failure to discern presence or absence of a line coloring), even if the line coloring is in a colored state that can be read (or that allows for discerning of presence or absence of the line coloring) by visual checking by humans.

In addition, when attempting to read a line coloring on a chromatographic strip from a photograph taken by a device including, for example, an image sensor, the photograph may have been affected by a phenomenon generally referred to as an “overexposed highlight” under the effect of light from, for example, an illuminator during photographing of the line coloring. Hence, a weaker line coloring may be read with a poorer accuracy.

Moreover, because a line coloring (color development on a test line) on a chromatographic strip may discolor (fade or deepen) over time, it is difficult to store a chromatographic strip, which has been through testing, for a long term while keeping it in the exact line coloring state as during the testing. Hence, a long-term storage of information regarding a line coloring on a chromatographic strip, which has been through testing, has a problem that the color-developed state on the chromatographic strip may change and the information representing the exact line coloring during the testing may be lost when the storage is by storage of the chromatographic strip itself, or a problem that it is difficult to discern a test line having a weak color development from a captured image of the chromatographic strip when the storage is by storage of the captured image.

As revealed by the recent years' worldwide COVID-19 pandemic problem, there is a high necessity for provision and improvement of a system that can, for example, collect and analyze information that enables determination of a result of analyte determination.

The present invention aims for solving the various problems in the related art and achieving an object described below. That is, an object of the present invention is to provide, for example, an automatic determination system that is capable of storing information that enables discerning of expression information on a chromatographic strip that has been through testing, in the form of uniform information that can be stored for a long term, and is capable of accurately deriving a result of determination on the chromatographic strip based on the information that enables discerning of the expression information.

### Solution to Problem

Means for solving the above problems are as follows.
<1> An automatic determination system, including:
an information storage unit configured to store result-of-detection image information acquired from a chromatographic strip support tool carrying the result-of-detection image information, the result-of-detection image information representing transcribed information regarding expression information obtained on a chromatographic strip that has been through testing; and
a result-of-determination deriving unit configured to check an image included in the result-of-detection image information against a data table, and derive a result of determination included in the data table and corresponding to the image.
<2> The automatic determination system according to <1>,
wherein the information storage unit is configured to store analyte information in association with the result-of-detection image information, the analyte information being acquired from the chromatographic strip support tool further carrying the analyte information, the analyte information being regarding an analyte supplied to the chromatographic strip.
<3> The automatic determination system according to <2>,
wherein the information storage unit is configured to store the result of determination derived by the result-of-determination deriving unit in association with the analyte information and the result-of-detection image information.
<4> The automatic determination system according to <2> or <3>,
wherein the analyte information is code information, and the result-of-detection image information is or scantron information.
<5> The automatic determination system according to any one of <2> to <4>, further including:
an information acquiring unit configured to acquire either or both of the analyte information and the result-of-detection image information.
<6> The automatic determination system according to <5>, wherein the information acquiring unit is an image capturing unit.
<7> An automatic determination method, including:
an information storing step of storing result-of-detection image information acquired from a chromatographic strip support tool carrying the result-of-detection image information, the result-of-detection image information representing transcribed information regarding expression information obtained on a chromatographic strip that has been through testing; and
a result-of-determination deriving step of checking an image included in the result-of-detection image information against a data table, and deriving a result of determination included in the data table and corresponding to the image.
<8> An automatic determination program causing a computer to execute:
an information storing process of storing result-of-detection image information acquired from a chromatographic strip support tool carrying the result-of-detection image information, the result-of-detection image information representing transcribed information regarding expression information obtained on a chromatographic strip that has been through testing; and
a result-of-determination deriving process of checking an image included in the result-of-detection image information against a data table, and deriving a result of determination included in the data table and corresponding to the image.

### Advantageous Effects of Invention

The present invention can provide, for example, an automatic determination system that is capable of storing information that enables discerning of expression information on a chromatographic strip that has been through testing, in the form of uniform information that can be stored for a long term, and is capable of accurately deriving a result of determination on the chromatographic strip based on the information that enables discerning of the expression information.

### BRIEF DESCRIPTION OF DRAWINGS

FIG. 1A is a view illustrating an example of how a line coloring on a chromatographic strip is read with a terminal device including an image sensor (which is an example of an information acquiring unit) according to an existing technique.

FIG. 1B is a view illustrating an example of how a line coloring on a chromatographic strip is read with a terminal device including an image sensor (which is an example of an information acquiring unit) according to an existing technique.

FIG. 1C is a view illustrating an example of how a line coloring on a chromatographic strip is read with a terminal device including an image sensor (which is an example of an information acquiring unit) according to an existing technique.

FIG. 2 is a view illustrating an example of a data table used for deriving a result of determination.

FIG. 3 is a view illustrating an example of a result-of-determination table in which a result of determination can be stored in association with analyte information and result-of-detection image information.

FIG. 4 is an oblique view illustrating an example of a chromatographic strip support tool and an example of a chromatographic strip according to a first embodiment.

FIG. 5 is a schematic view illustrating an example of a display section when a chromatographic strip is placed on a support surface such that an extremity of the chromatographic strip abuts against a first abutting portion.

FIG. 6 is a plan view illustrating an example of a chromatographic strip support tool according to a first embodiment.

FIG. 7A is a cross-sectional view of FIG. 6 along an X-X′ axis seen from a viewing location A in FIG. 6.

FIG. 7B is a cross-sectional view of FIG. 6 along a Y-Y′ axis seen from a viewing location B in FIG. 6.

FIG. 8A is an end surface view seen from a viewing location A in FIG. 6.

FIG. 8B is an end surface view seen from a viewing location B in FIG. 6.

FIG. 8C is an end surface view seen from a viewing location C in FIG. 6.

FIG. 9A is an oblique view illustrating an example of a chromatographic strip support tool according to a first embodiment, including a support surface 11 and a foldable portion 22 continuous with the support surface 11 via a folding line 21.

FIG. 9B is an oblique view illustrating an example of a chromatographic strip support tool according to a first embodiment in a state of a foldable portion 22 being folded along a folding line 21.

FIG. 9C is an oblique view illustrating an example of a chromatographic strip support tool according to a first embodiment in a state of a foldable portion 22 being fixed in a manner to abut against a support surface 11.

FIG. 10A is a schematic view illustrating an example of a method of using a chromatographic strip support tool according to a first embodiment.

FIG. 10B is a schematic view illustrating an example of a method of using a chromatographic strip support tool according to a first embodiment.

FIG. 10C is a schematic view illustrating an example of a method of using a chromatographic strip support tool according to a first embodiment.

FIG. 10D is a schematic view illustrating an example of a method of using a chromatographic strip support tool according to a first embodiment.

FIG. 11 is a schematic view illustrating an example of a display section 14 according to a modification of a first embodiment.

FIG. 12 is a schematic view illustrating an example of a display section 14 according to a modification of a first embodiment.

FIG. 13 is an oblique view illustrating an example of a chromatographic strip support tool and an example of a chromatographic strip according to a second embodiment.

FIG. 14 is a plan view illustrating an example of a chromatographic strip support tool according to a second embodiment.

FIG. 15A is a cross-sectional view of FIG. 14 along an X-X′ axis seen from a viewing location A in FIG. 14.

FIG. 15B is a cross-sectional view of FIG. 14 along a Y-Y′ axis seen from a viewing location B in FIG. 14.

FIG. 16A is an end surface view seen from a viewing location A in FIG. 14.

FIG. 16B is an end surface view seen from a viewing location B in FIG. 14.

FIG. 16C is an end surface view seen from a viewing location C in FIG. 14.

FIG. 17 is an oblique view illustrating an example of a chromatographic strip support tool and an example of a chromatographic strip according to a third embodiment.

FIG. 18 is a plan view illustrating an example of a chromatographic strip support tool according to a third embodiment.

FIG. 19A is a cross-sectional view of FIG. 18 along an X-X′ axis seen from a viewing location A in FIG. 18.

FIG. 19B is a cross-sectional view of FIG. 18 along a Y-Y′ axis seen from a viewing location B in FIG. 18.

FIG. 20A is an end surface view seen from a viewing location A in FIG. 18.

FIG. 20B is an end surface view seen from a viewing location B in FIG. 18.

FIG. 20C is an end surface view seen from a viewing location C in FIG. 18.

FIG. 21 is a plan view illustrating an example of the styles in which various kinds of information carried on a chromatographic strip support tool are displayed according to a third embodiment.

FIG. 22 is a plan view illustrating another example of the styles in which various kinds of information carried on a chromatographic strip support tool are displayed according to a third embodiment.

FIG. 23 is a block diagram illustrating an example of a configuration of an automatic determination system according to an embodiment of the present invention.

FIG. 24 is a block diagram illustrating an example of a hardware configuration of an automatic determination system according to an embodiment of the present invention.

FIG. 25 is a block diagram illustrating an example of a functional configuration of an information storage unit of an automatic determination system according to an embodiment of the present invention.

FIG. 26 is a block diagram illustrating an example of a functional configuration of a result-of-determination deriving unit of an automatic determination system according to an embodiment of the present invention.

FIG. 27 is a block diagram illustrating an example of a hardware configuration of a terminal device of an automatic determination system according to an embodiment of the present invention.

FIG. 28 is a block diagram illustrating an example of a functional configuration of a terminal device of an automatic determination system according to an embodiment of the present invention.

FIG. 29 is a flowchart illustrating an example of a flow of automatic determination on a chromatographic strip that has been through testing, using an automatic determination system of the present invention.

### DESCRIPTION OF EMBODIMENTS

### (Automatic Determination System)

An automatic determination system of the present invention is based on the present inventors' finding that existing techniques cannot store information that enables discerning of expression information on a chromatographic strip that has been through testing, for a long term, and may not be able to accurately derive a result of determination on the chromatographic strip.

As described above, a line coloring (color development on a test line) on a chromatographic strip may be sometimes weak (or light) because the intensity of the line coloring is dependent on the abundance or concentration of the target substance in a supplied analyte. In this case, even if the line coloring has a density at which the line coloring can be correctly read by visual checking by humans, the line coloring may not be correctly read (i.e., mistaken determination may occur due to failure to discern presence or absence of a line coloring) if it is read in an automated manner using a device including, for example, an image sensor.

In addition, as described above, when attempting to read a line coloring on a chromatographic strip from a photograph of the line coloring taken by a device including, for example, an image sensor, the photograph may have been affected by a phenomenon generally referred to as an “overexposed highlight” under the effect of light from, for example, an illuminator during photographing of the line coloring. Hence, a weaker line coloring may be read with a poorer accuracy.

For this reason, when reading a line coloring on a chromatographic strip (or discerning whether a test line has developed a color) based on an image of the test line itself on the chromatographic strip captured using, for example, an image sensor, the line coloring may not be read correctly and mistaken determination may occur if the line coloring is weak (light).

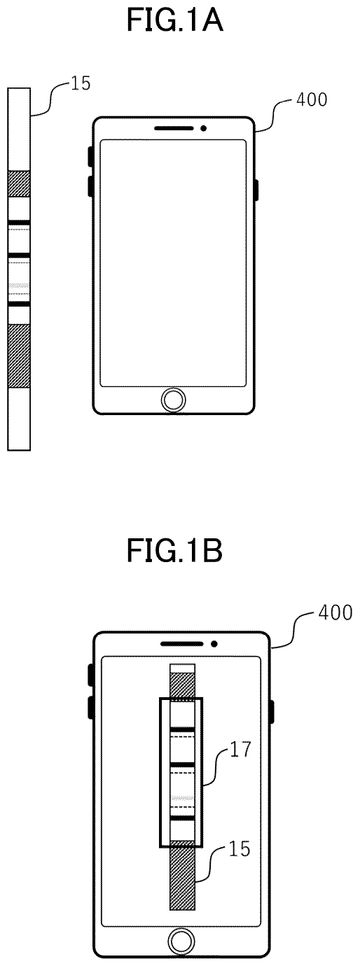

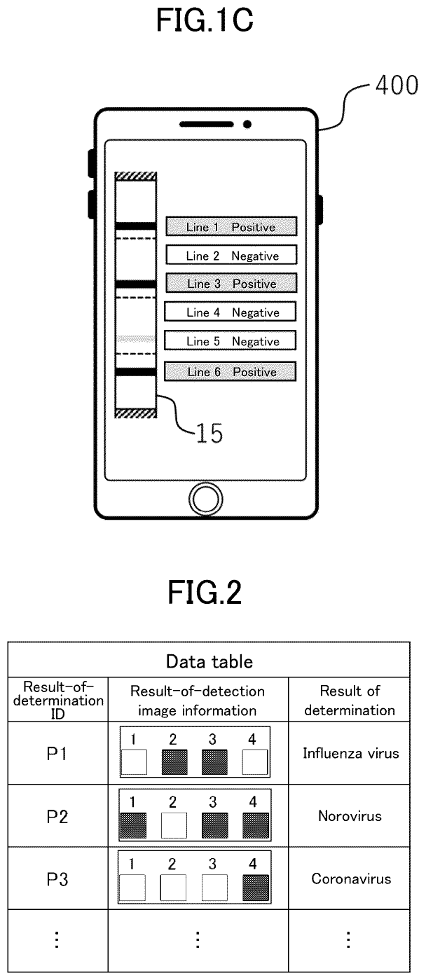

FIG. 1A to FIG. 1C are views illustrating an example of how a line coloring on a chromatographic strip is read with a terminal device including an image sensor (which is an example of an information acquiring unit) according to an existing technique.

According to the existing technique, as illustrated in FIG. 1A, a chromatographic strip 15 that has been through testing is prepared, and a terminal device 400 (e.g., a smartphone) is started to photograph the chromatographic strip 15.

Then, according to the existing technique, as illustrated in FIG. 1B, any line coloring in a membrane section 17 in which an analyte is developed is read and discerned from the chromatographic strip 15 photographed using the terminal device 400.

Then, according to the existing technique, as illustrated in FIG. 1C, presence or absence of a line coloring (i.e., positive or negative) is determined based on the result of reading any line coloring. The example illustrated in FIG. 1C is an example where a line 5, which is colored more lightly than other detected lines 1, 3, and 6, cannot be correctly read with the terminal device 400, although coloring of the line 5 can be discerned by visual checking. In the example illustrated in FIG. 1C, even though the line 5 is actually colored, the line 5 cannot be read correctly with the terminal device 400 and determined mistakenly as negative.

Moreover, as described above, the intensity of a line coloring (color development on a test line) on a chromatographic strip may change over time (to fade or deepen). Therefore, when a chromatographic strip itself, which has been through testing, is stored for the purpose of long-term storage of information regarding a line coloring on the chromatographic strip, the information representing the exact line coloring during the testing may become lost the instant the color development on the test line fades. Moreover, as described above, when a captured image of a chromatographic strip that has been through testing is stored for the purpose of long-term storage of information regarding a line coloring on the chromatographic strip, it may be difficult to discern a test line having a weak color development (i.e., a light line coloring) from the image.

Hence, the existing technique is problematic in that information that enables discerning of expression information on a chromatographic strip that has been through testing (e.g., information regarding which test line has developed a color) may not be stored for a long term. The existing technique is also problematic in that device reading of a line coloring on a chromatographic strip that has been through testing may fail to correctly read the line coloring, and it may be impossible to accurately derive a result of determination (i.e., mistaken determination may occur).

Here, the present inventors have obtained the following findings as a result of earnest studies into, for example, an automatic determination system that is capable of storing information that enables discerning of expression information on a chromatographic strip that has been through testing, in the form of uniform information that can be stored for a long term, and is capable of accurately deriving a result of determination on the chromatographic strip based on the information that enables discerning of the expression information.

That is, the present inventors have found it possible to store information that enables discerning of expression information on a chromatographic strip that has been through testing, in the form of uniform information that can be stored for a long term and to accurately derive a result of determination on the chromatographic strip based on the information that enables discerning of the expression information, by means of, for example, an automatic determination system including: an information storage unit configured to store result-of-detection image information acquired from a chromatographic strip support tool carrying the result-of-detection image information, the result-of-detection image information representing transcribed information regarding expression information obtained on a chromatographic strip that has been through testing; and a result-of-determination deriving unit configured to check an image included in the result-of-detection image information against a data table, and derive a result of determination included in the data table and corresponding to the image.

The information storage unit of the automatic determination system of the present invention stores result-of-detection image information acquired from a chromatographic strip support tool. The chromatographic strip support tool carries the result-of-detection image information representing transcribed information regarding expression information obtained on a chromatographic strip that has been through testing. That is, the automatic determination system of the present invention stores result-of-detection image information representing transcribed information regarding expression information obtained on a chromatographic strip that has been through testing.

Here, the expression information obtained on a chromatographic strip that has been through testing may be, for example, information regarding a biomolecule that is assumed to be contained in an analyte supplied to the chromatographic strip and that corresponds to the location of a line coloring induced by the analyte in the testing using the chromatographic strip. In other words, information regarding the expression information obtained on a chromatographic strip that has been through testing may be, for example, information regarding a detection item that has a line coloring that has occurred in the testing using the chromatographic strip.

As the result-of-detection image information, it is possible to use, for example, image information representing information transcribed to a chromatographic strip support tool regarding a detection item (expression information) that has a line coloring that has occurred as mentioned. As the result-of-detection image information representing the transcribed information regarding the expression information, it is possible to use, for example, information representing a marking (or a sign) that: is given at such a location on a chromatographic strip support tool as corresponding to a detection item having obtained expression information; is captured as an image; and is changed to image information, by a person, who handles the process, and reads the expression information obtained on a chromatographic strip that has been through testing.

As can be seen, the automatic determination system of the present invention stores the result-of-detection image information representing transcribed information regarding expression information obtained on a chromatographic strip that has been through testing. Hence, for example, even when a line coloring on the chromatographic strip discolors over time or the line coloring is light, the expression information based on the line coloring can be stored for a long term (preserved for a long term) in the form of result-of-detection image information representing, for example, a marking by which the expression information is transcribed, i.e., in the form of information that enables discerning of the expression information.

That is, the automatic determination system of the present invention can store information that is for discerning (or picking out) expression information, in the form of result-of-detection image information including information representing the expression information in a transcribed form embodied by, for example, a marking. Hence, by transcribing expression information as image information that does not change over time and can be stored for a long term, it is possible to reduce the effects of the lighting environment that may be imposed on reading with, for example, an image sensor when a line coloring itself is weak (light), and to discern (pick out) the expression information accurately.

In addition, the automatic determination system of the present invention can store, for example, result-of-detection image information that represents transcribed information in a desired form. Hence, it is possible to store the information that enables discerning of expression information on a chromatographic strip that has been through testing, in the form of uniform information.

Moreover, the information storage unit of the automatic determination system of the present invention checks an image included in the result-of-detection image information against a data table, and derives a result of determination included in the data table and corresponding to the image. That is, the automatic determination system of the present invention derives (finds) a result of determination on a chromatographic strip that has been through testing, based on the data table in which, for example, image information including information representing transcribed information regarding expression information and a result of determination (result of testing) on a chromatographic strip are associated with each other.

In this way, the automatic determination system of the present invention derives a result of determination on a chromatographic strip based on the result-of-detection image information representing expression information transcribed as, for example, a marking. Therefore, even when a line coloring itself is weak (light), it is possible to read the line coloring correctly based on the transcribed information and to accurately derive a result of determination (result of testing) on the chromatographic strip.

Hence, the automatic determination system of the present invention including the information storage unit and the result-of-determination deriving unit can store information that enables discerning of expression information on a chromatographic strip that has been through testing, in the form of uniform information that can be stored for a long term, and can accurately derive a result of determination on the chromatographic strip based on the information that enables discerning of the expression information.

Hence, use of the automatic determination system of the present invention enables appropriate storage of information regarding expression information obtained on a chromatographic strip that has been through testing, and automatic, accurate derivation and determination of a result of testing using the chromatographic strip.

In the following description, the details of, for example, each unit of the automatic determination system of the present invention will be described.

The automatic determination system of the present invention includes an information storage unit and a result-of-determination deriving unit, preferably includes an information acquiring unit, and further includes other units as needed. The respective units of the automatic determination system of the present invention may be independent (separate from each other). When the respective units are independent, for example, it is preferable to connect the respective units using a network such as an intranet and the Internet. In other words, in the automatic determination system of the present invention, it is preferable that the information storage unit and the result-of-determination deriving unit be connected to each other via a network.

### <Information Storage Unit>

The information storage unit is a unit configured to store result-of-detection image information acquired from a chromatographic strip support tool carrying the result-of-detection image information, the result-of-detection image information representing transcribed information regarding expression information obtained on a chromatographic strip that has been through testing.

The information storage unit is not particularly limited and may be appropriately selected in accordance with the intended purpose so long as the information storage unit can store (memorize) result-of-detection image information. It is possible to realize the information storage unit using, for example, a publicly known computer, a publicly known server device, and a publicly known portable terminal.

### <Result-of-Detection Image Information>>

The result-of-detection image information stored in the information storage unit is not particularly limited and may be appropriately selected in accordance with the intended purpose, so long as the result-of-detection image information is image information representing transcribed information regarding expression information obtained on a chromatographic strip that has been through testing, and acquired from a chromatographic strip support tool. That is, the result-of-detection image information includes, for example, an image representing transcribed information regarding expression information.

Here, as described above, as the result-of-detection image information, it is possible to use, for example, information representing a marking (or a sign) that: is given at such a location on a chromatographic strip support tool as corresponding to a detection item having obtained expression information; is captured as an image; and is changed to image information, by a person who handles the process, and reads the expression information obtained on a chromatographic strip that has been through testing.

The expression information is not particularly limited and may be appropriately selected in accordance with the intended purpose so long as the expression information is information regarding a biomolecule that is expressed (detected). Moreover, as described above, the expression information may be, for example, information (e.g., name) regarding a biomolecule that is assumed to be contained in an analyte supplied to a chromatographic strip and that corresponds to the location of a line coloring induced by the analyte in a testing using the chromatographic strip. What is induced by the analyte supplied to a chromatographic strip is not particularly limited to a line coloring, and may be, for example, a coloring in a dot shape and a coloring in a circular shape, or may be, a color fading (i.e., a reaction to become lighter than the average color of a chromatographic strip).

As the result-of-detection image information, it is possible to use, for example, expression information that is read and transcribed to a scantron region of a chromatographic strip support tool by solid fill-out or by marking by, for example, a checkmark by the person handling the process (i.e., image information representing a captured image of the scantron region in which a location (checkbox) corresponding to a detection item, which has the obtained expression information, is solidly filled out). That is, in other words, in the automatic determination system of the present invention, it is preferable that the result-of-detection image information be scantron information representing a captured image of a scantron region on a chromatographic strip support tool.

In this case, the automatic determination system of the present invention can store information representing expression information in a more easy-to-read and more uniform form, and can derive a result of determination (result of testing) on a chromatographic strip more easily and more accurately.

To obtain the result-of-detection image information, the method for giving a marking (or a sign) to such a location on a chromatographic strip support tool as corresponding to a detection item (expression information), which has obtained expression information, is not particularly limited to the aforementioned method of solidly filling out a checkbox or giving a checkmark to a checkbox of a scantron. The method is not particularly limited and may be appropriately selected in accordance with the intended purpose, so long as it is possible to give a sign that can be discerned by an image process.

<<Analyte information>>

In the automatic determination system of the present invention, it is preferable that the information storage unit further stores information (analyte information) regarding an analyte supplied to a chromatographic strip.

More specifically, in the automatic determination system of the present invention, it is preferable that the information storage unit stores analyte information acquired from a chromatographic strip support tool further carrying the analyte information regarding an analyte supplied to a chromatographic strip.

The analyte information is not particularly limited and may be appropriately selected in accordance with the intended purpose so long as the analyte information is information regarding an analyte supplied to a chromatographic strip. Examples of the analyte information include information regarding the subject who has donated the analyte, information indicating the date on which the analyte is collected, and information regarding, for example, the hospital at which the analyte is collected. Examples of the information regarding the subject who has donated the analyte include identification information (ID information) of the subject, information of the date and year of birth of the subject, information of the name of the subject, information of the age of the subject, information of the gender of the subject, information of the blood type of the subject, and information of the affiliation to which the subject belongs to.

The analyte information may be information displayed (written) on the chromatographic strip support tool. The style in which the analyte information is displayed (written) on the chromatographic strip support tool is not particularly limited and may be appropriately selected in accordance with the intended purpose. Examples of the style include a display style in letters, a display style in symbols, and a display style in patterns.

Moreover, it is preferable to display the analyte information in a display style in symbols or patterns among the display (writing) styles mentioned above. More specifically, it is preferable to display the analyte information in the form of code information represented by combination of symbols or patterns. In other words, in the present invention, it is preferable that the analyte information be code information. Examples of code information include a barcode and a two-dimensional code (QR code (registered trademark)).

When the analyte information is code information, a large quantity of information regarding the analyte can be displayed even when, for example, the region of the chromatographic strip support tool in which the analyte information can be displayed is limited. Moreover, when the analyte information is code information, the analyte information can be read easily and accurately by, for example, an image capturing unit.

The analyte information may be information that is not displayed (written) on the chromatographic strip support tool. More specifically, the analyte information may be displayed in the form of electronic information that can be read out. Examples of the electronic information include a Radio Frequency Identification (RFID) tag, and an Integrated Circuit (IC) tag. In this case, it is preferable that the electronic information be internally stored in the chromatographic strip support tool.

When the analyte information is electronic information, a large quantity of information regarding the analyte can be displayed (read out) even when, for example, the region of the chromatographic strip support tool in which the analyte information can be displayed is extremely limited. Moreover, when the analyte information is electronic information, the analyte information can be rewritten repeatedly an indefinite number of times, and a chromatographic strip support tool that has been used once can be re-used.

A chromatographic strip support tool may include information other than the result-of-detection image information and the analyte information described above, and may include, for example, product identification information of the chromatographic strip support tool, and information regarding how to use the chromatographic strip support tool. Examples of the style in which these kinds of information are displayed (written) include a display style in letters, a display style in symbols, and a display style in patterns, like the analyte information. When these kinds of information are not displayed (written) on a chromatographic strip support tool, they may be displayed (read out) in the form of electronic tag information, like the analyte information.

For example, it is preferable that the result-of-detection image information and the analyte information carried on a chromatographic strip support tool, which are to be stored in the information storage unit, be information acquired by an information acquiring unit described below. However, they may be information manually input by the person handling the process or may be information downloaded from, for example, any other computer (any other database).

Here, in the automatic determination system of the present invention, it is preferable that the information storage unit stores the analyte information and the result-of-detection image information in association (while being linked) with each other. In the automatic determination system of the present invention, it is preferable that the information storage unit stores in association with each other, the analyte information and the result-of-detection image information that are acquired from a chromatographic strip support tool further carrying the analyte information regarding an analyte supplied to a chromatographic strip.

In this case, the automatic determination system of the present invention can store the analyte information regarding an analyte supplied to a chromatographic strip and information regarding expression information obtained on the chromatographic strip that has been through testing in association with each other in the form of uniform information that can be stored for a long term, and can use the stored information for automatic determination.

It is preferable that the information storage unit stores the various kinds of information, which are carried on a chromatographic strip support tool, such as the analyte information and the result-of-detection image information described above, in the form of a database. It is also preferable that the information storage unit can renew (update) the various kinds of information, which are carried on a chromatographic strip support tool, such as the analyte information and the result-of-detection image information described above, in the database anytime.

Specific embodiments of, for example, the analyte information and transcribed information regarding expression information, which are carried on a chromatographic strip support tool, will be described below.

### <Result-of-Determination Deriving Unit>

The result-of-determination deriving unit is a unit configured to check an image included in the result-of-detection image information against a data table, and derive a result of determination included in the data table and corresponding to the image.

The result-of-determination deriving unit is not particularly limited and may be appropriately selected in accordance with the intended purpose so long as the result-of-determination deriving unit can derive a result of determination. It is possible to realize the result-of-determination deriving unit by using, for example, a publicly-known computer, a publicly-known server device, and a publicly-known portable terminal.

Here, as described above, the result-of-determination deriving unit derives (finds) a result of determination on a chromatographic strip that has been through testing, based on a data table in which, for example, image information including transcribed information regarding expression information and a result of determination (result of testing) on a chromatographic strip are associated with each other.

The data table used for deriving a result of determination is not particularly limited and may be appropriately selected in accordance with the intended purpose so long as image information including transcribed information regarding expression information and a result of determination (result of testing) on a chromatographic strip are associated with each other in the data table. As the data table used for deriving a result of determination, it is possible to use, for example, a data table in which, for each arrangement pattern that may be assumed by transcribed information regarding expression information in the image of the result-of-detection image information, a result of determination on a chromatographic strip is associated with the arrangement pattern in a manner that checking against the arrangement pattern is possible.

More specifically, when the result-of-detection image information is, for example, scantron information representing a captured image of a scantron region of a chromatographic strip support tool, as the data table, it is possible to use a data table in which, for each fill-out pattern (i.e., a pattern to be formed depending on which item is filled out) in the scantron information, a result of determination (result of testing) corresponding to the fill-out pattern is associated with the fill-out pattern in a manner that checking against the fill-out pattern is possible.

FIG. 2 is a view illustrating an example of a data table used for deriving a result of determination. As the data table used for deriving a result of determination, it is possible to use, for example, a data table in which a result-of-determination ID, result-of-detection image information, and a result of determination are recorded as illustrated in FIG. 2. As the data table used for deriving a result of determination, it is possible to use, for example, a data table in which a result-of-determination ID, result-of-detection image information, and a result of determination are recorded in one record (row), and different kinds of information regarding the result of determination are recorded column by column as illustrated in FIG. 2.

In the example illustrated in FIG. 2, for example, in the record (row) indexed with a result-of-determination ID “P1”, result-of-detection image information indicating that Item 2 and Item 3 are detected, and information indicating that the result of determination corresponding to this result-of-detection image information is “influenza virus” are recorded. The result-of-determination deriving unit derives a result of determination included in the data table and corresponding to an image included in result-of-detection image information representing transcribed information regarding expression information obtained on a chromatographic strip that has been through testing, by, for example, performing an image process such as pattern matching between the result-of-detection image information included in the data table as illustrated in FIG. 2 and the concerned result-of-detection image information representing transcribed information regarding expression information obtained on the chromatographic strip that has been through testing.

Moreover, in the automatic determination system of the present invention, it is preferable that the information storage unit stores a result of determination derived by the result-of-determination deriving unit in association with analyte information and result-of-detection image information. In other words, in the automatic determination system of the present invention, it is preferable that, for example, a result of determination derived based on result-of-detection image information be stored in association (while being linked) with the concerned result-of-detection image information and analyte information.

In this way, the automatic determination system of the present invention can store analyte information regarding an analyte supplied to a chromatographic strip, information regarding expression information obtained on the chromatographic strip that has been through testing, and a result of determination (result of testing) on the chromatographic strip that has been through testing in association with one another in the form of uniform information that can be stored for a long term.

When no result of determination corresponding to an image included in result-of-detection image information is included in the data table, the result-of-determination deriving unit may add a new result of determination corresponding to the image in the data table together with the image. Moreover, when no result of determination corresponding to an image included in result-of-detection image information is included in the data table, the result-of-determination deriving unit may deduct and derive a result of determination based on the results of determination included in the data table.

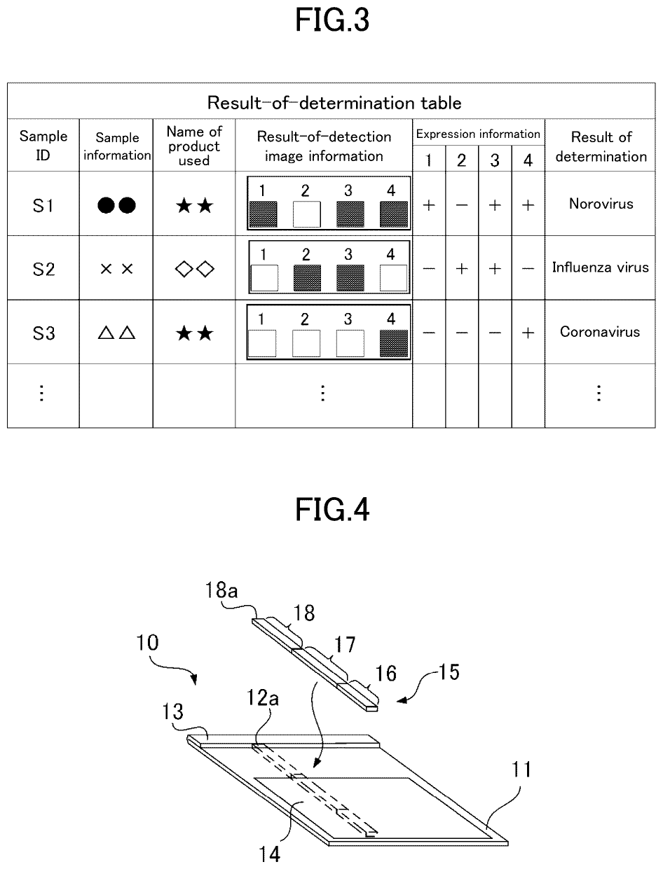

FIG. 3 is a view illustrating an example of a result-of-determination table in which a result of determination can be stored in association with analyte information and result-of-detection image information. As the result-of-determination table, it is possible to use, for example, a table in which a sample (analyte) ID, sample (analyte) information, name of product used, result-of-detection image information, expression information, and a result of determination are recorded as illustrated in FIG. 3. As the result-of-determination table, it is possible to use, for example, a table in which a sample (analyte) ID, sample (analyte) information, name of product used, result-of-detection image information, expression information, and a result of determination are recorded in one record (row) and different kinds of information regarding a result of determination are recorded column by column as illustrated in FIG. 3.

In the example illustrated in FIG. 3, for example, in a record (row) indexed with a sample ID “S1”, an item that is read from result-of-detection image information as being detected (i.e., an item that is read as a biomolecule being expressed) on a chromatographic strip that has been through testing is denoted by “+” and an item that is read as not being detected is denoted by “−” and recorded in this manner in the column “Expression information”.

### <Information Acquiring Unit>

The information acquiring unit is a unit configured to acquire either or both of analyte information and result-of-detection image information.

The information acquiring unit is not particularly limited and may be appropriately selected in accordance with the intended purpose, so long as the information acquiring unit can acquire either or both of analyte information and result-of-detection image information. Examples of the information acquiring unit include an image capturing unit and a code reader.

Examples of the image capturing unit include a device including a camera (image sensor), and a scanner. Examples of the device including a camera (image sensor) include terminal devices such as a smartphone, a tablet terminal, and a personal computer (PC).

Examples of the code reader include a barcode reader and a two-dimensional code reader. As the code reader, a terminal device in which an application that enables reading of, for example, a barcode and a two-dimensional code is installed may be also be used.

Examples of electronic information reader/writer include a RFID reader/writer and an IC reader/writer. As the electronic information reader/writer, a terminal device in which an application that enables either or both of reading of and writing on of, for example, an RFID tag and an IC tag is installed may also be used.

As the information acquiring unit, for example, an information acquiring unit configured to acquire analyte information and an information acquiring unit configured to acquire result-of-detection image information may be provided independently. However, it is preferable to use a unit that can acquire analyte information and result-of-detection image information collectively. As an information acquiring unit that can acquire analyte information and result-of-detection image information collectively, for example, the aforementioned terminal device such as a smart-phone can be suitably used.

When storing information acquired by the information acquiring unit in the information storage unit, for example, it is preferable that the information acquiring unit sends the information acquired by the information acquiring unit to the information storage unit. In other words, in the automatic determination system of the present invention, it is preferable that the information storage unit and the information acquiring unit be communicably connected to each other.

The method by which the information acquiring unit sends analyte information and result-of-detection image information to the information storage unit is not particularly limited and may be appropriately selected in accordance with the intended purpose. For example, a method of the information acquiring unit sending analyte information and result-of-detection image information to the information storage unit via a network such as the Internet may be used. Hence, in the present invention, a terminal device such as a smartphone can be suitably used as the information acquiring unit in terms communicability with the information storage unit.

The method by which the information acquiring unit acquires either or both of analyte information and result-of-detection image information carried on a chromatographic strip support tool is not particularly limited and may be appropriately selected in accordance with the intended purpose. When the image acquiring unit is an image capturing unit, for example, it is preferable to capture an image of the whole of a chromatographic strip support tool, and locate and acquire analyte information (e.g., code information) and result-of-detection image information (e.g., scantron information) that are included in the captured image.

When capturing an image of the whole of a chromatographic strip support tool as in the mentioned example, for example, it is possible to improve the accuracy in acquiring these kinds of information, by providing a marker for positioning and orienting at a predetermined location of the chromatographic strip support tool, determining the position and orientation of the chromatographic strip support tool in the captured image based on the marker, and locating and acquiring analyte information and result-of-detection image information. For example, when the planer shape of a chromatographic strip support tool is a quadrangular shape, it is preferable to provide the marker for positioning and orienting of the chromatographic strip support tool, at the four corners of the chromatographic strip support tool.

### <Other Units>

The other units are not particularly limited and may be appropriately selected in accordance with the intended purpose.

### (Automatic Determination Method)

An automatic determination method of the present invention includes:
an information storing step of storing result-of-detection image information acquired from a chromatographic strip support tool carrying the result-of-detection image information, the result-of-detection image information representing transcribed information regarding expression information obtained on a chromatographic strip that has been through testing; and
a result-of-determination deriving step of checking an image included in the result-of-detection image information against a data table, and deriving a result of determination included in the data table and corresponding to the image.

The automatic determination method of the present invention can be realized by, for example, the automatic determination system described above. Hence, a preferred embodiment of the automatic determination method of the present invention can be the same as that of the automatic determination system described above.

(Automatic Determination Program)

An automatic determination program of the present invention causes a computer to execute: an information storing process of storing result-of-detection image information acquired from a chromatographic strip support tool carrying the result-of-detection image information, the result-of-detection image information representing transcribed information regarding expression information obtained on a chromatographic strip that has been through testing; and a result-of-determination deriving process of checking an image included in the result-of-detection image information against a data table, and deriving a result of determination included in the data table and corresponding to the image.

For example, the automatic determination program of the present invention may be a program that causes a computer to realize the automatic determination method described above. In other words, by causing a computer to execute the automatic determination program of the present invention, it is possible to cause the computer to function as the automatic determination system described above. Hence, a preferred embodiment of the automatic determination program of the present invention can be the same as that of the automatic determination system and the automatic determination method described above.

It is possible to create the automatic determination program of the present invention using various programming languages in accordance with the configuration of the computer to be used and the type and version of the operating system.

The automatic determination program of the present invention may be recorded on a memory medium such as a hard disk, or may be recorded on a memory medium such as a Compact Disc-Read Only Memory (CD-ROM), a Digital Versatile Disc-ROM (DVD-ROM), a Magneto-Optical (MO) disk, a Universal Serial Bus (USB) memory, a Secure Digital (SD) card, a micro SD card, and a compact flash (registered trademark).

The automatic determination program of the present invention may also be recorded in an external memory area (e.g., a computer) that can be accessed by another computer via an information communication network. In this case, the automatic determination program of the present invention recorded in the external memory area can be used as needed by being installed on a hard disk from the external memory area via the information communication network.

The automatic determination program of the present invention may be recorded on a plurality of memory media while being divided into desirably selected processes.

### <Embodiments of Chromatographic Strip Support Tool>

The embodiments of a chromatographic strip support tool that can be used in the present invention will be described below with reference to the drawings.

### First Embodiment

An example of a chromatographic strip support tool according to the first embodiment will be described with reference to FIG. 4 to FIG. 8C.

In the example illustrated in FIG. 4 and FIG. 5, a chromatographic strip 15 includes an analyte supply section 16 to which an analyte is supplied, a membrane section 17 in which the analyte is developed, and an absorption pad section 18 configured to absorb the analyte. After an analyte is supplied to the analyte supply section 16 and developed with a developing solution, a line coloring 19b (color development on a test line), which occurs when a predetermined biological component is present, may become readable in the membrane section 17. In this way, the biological component contained in the supplied analyte can be detected.

A line coloring (color development on a test line) on the chromatographic strip may be exhibited by, for example, colloid particles formed of metals such as gold, silver, copper, and platinum, colored latexes obtained by coloring latexes with, for example, pigments and dyes, and silica nanoparticles obtained by immobilizing pigment molecules in silica particles. Among these, it is preferable to use colloid particles formed of gold (coloring in red), and colored latex particles formed of water-dispersible high-molecular-weight polymers colored in, for example, blue and red.

Examples of the chromatographic strip include a chromatographic strip for immunochromatography and a chromatographic strip for nucleic acid chromatography. As the chromatographic strip, a lateral flow type is preferable.

In accordance with the intended purpose, the chromatographic strip can be used for various detection uses that are not particularly limited. For example, the chromatographic strip can be suitably used for detection uses in which accuracy in line coloring reading (accuracy in determination) is particularly demanded, such as examination for infectious diseases, detection of allergens in foods, and food quality control (inspection for falsely labeled foods and genetically modified foods).

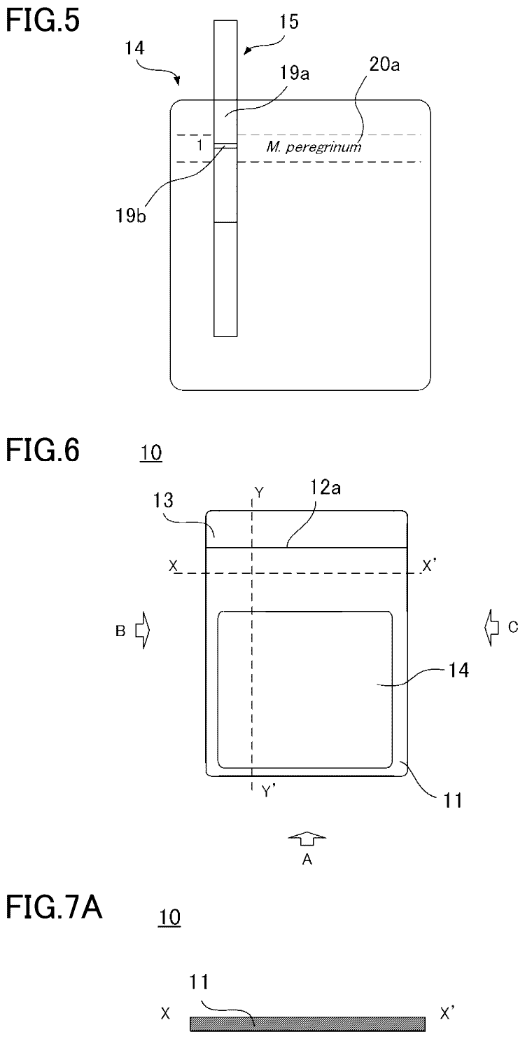

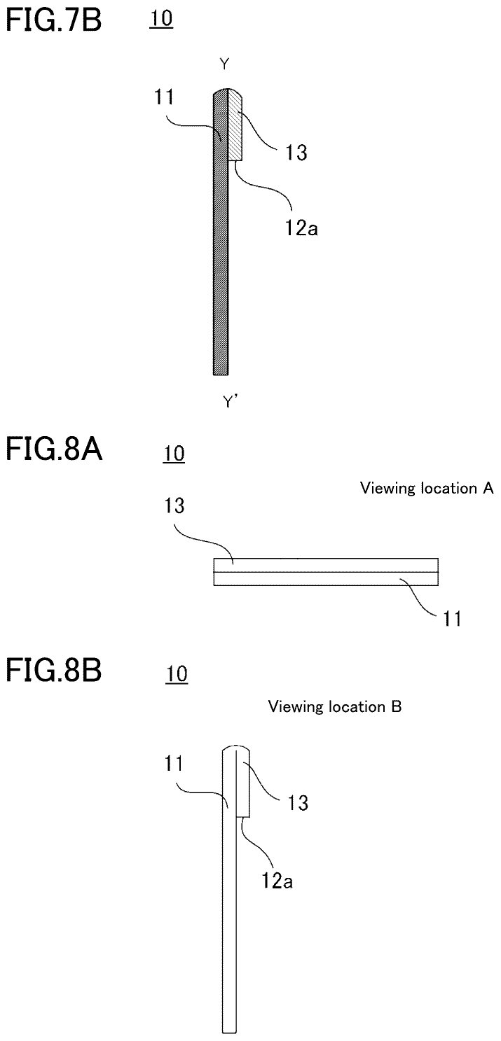

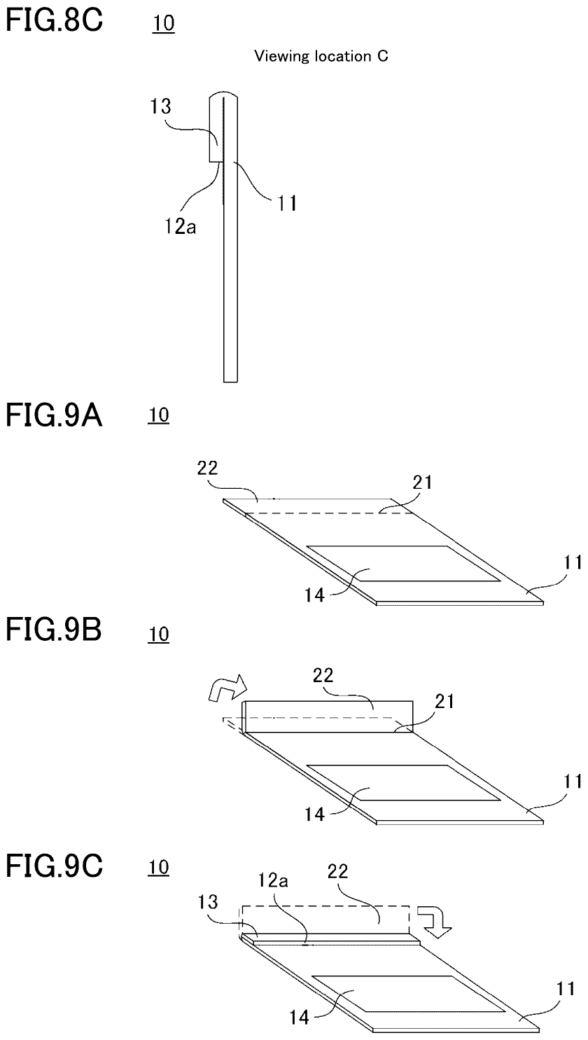

A chromatographic strip support tool 10 according to the first embodiment includes a support surface 11 that is continuous and on which the chromatographic strip 15 can be placed as illustrated in FIG. 4 and FIG. 6 to FIG. 8C, a projected portion 13 formed on the support surface 11 as illustrated in FIG. 4, FIG. 6, and FIG. 7B to FIG. 8C, and a display section 14 formed on the support surface 11 as illustrated in FIG. 2 to FIG. 4.

As the material of the support surface 11, the projected portion 13, and the display section 14, it is preferable to use paper that is coated with a water shedding material because such a material can inhibit soaking with an analyte.

The projected portion 13 has a first abutting portion 12a that can abut against an extremity 18a of the chromatographic strip 15 placed (put) on the support surface 11, the extremity 18a being present at one end side (absorption pad section 18) of the chromatographic strip 15, as illustrated in FIG. 4, FIG. 6, FIG. 7B, FIG. 8B, and FIG. 8C.

FIG. 5 is a schematic view illustrating an example of the display section 14 when the chromatographic strip 15 is placed (put) on the support surface 11 such that the extremity 18a of the chromatographic strip 15 abuts against the first abutting portion 12a.

The display section 14 has a detection item 20a as illustrated in FIG. 5. In FIG. 5, “M.peregrium” displayed as the detection item represents a kind of a fungus belonging to acid-fast bacilli.

The detection item 20a is displayed at a location that is the same as a predetermined location 19a of the chromatographic strip 15, the predetermined location 19a being a location at which a detection (line coloring 19b) may be observed.

The chromatographic strip support tool 10 includes a support surface 11 that is continuous and on which the chromatographic strip 15 can be placed, and a foldable portion 22 continuous with the support surface 11 via a folding line 21 as illustrated in FIG. 9A.

When the foldable portion 22 is folded along the folding line 21 as illustrated in FIG. 9B and is made to abut against the support surface 11 as illustrated in FIG. 9C, the foldable portion 22 becomes fixed in a manner to form the projected portion 13 having the first abutting portion 12a that can abut against the extremity 18a of the chromatographic strip 15 placed (put) on the support surface 11, the extremity 18a being present at the one end side (absorption pad section 18) of the chromatographic strip 15.

The foldable portion 22 is fixed by bonding with an adhesive agent.

Handlings and workings of the chromatographic strip support tool configured as described above will be described below with reference to FIG. 4 and FIG. 5.

When placing (putting) the chromatographic strip 15 on the support surface 11 of the chromatographic strip support tool 10, the chromatographic strip 15 is placed (put) on the support surface such that the extremity 18a of the chromatographic strip 15, present at the one end side (absorption pad section 18) opposite to the side at which the analyte supply section 16 is present, and the first abutting portion 12a abut against each other as illustrated in FIG. 4. This makes the detection item 20a of the display section 14 of the chromatographic strip support tool 10 be displayed at the same location as a detection (line coloring 19b) that may occur at the predetermined location 19a of the chromatographic strip 15 as illustrated in FIG. 5. As a result, it is possible to minimize occurrence of mistaken reading (mistaken detection) of a line coloring due to misplacement of the chromatographic strip 15.

The projected portion 13 abuts only against the absorption pad section 18 of the chromatographic strip 15 and does not abut against the membrane section 17. This makes it possible to obtain accurate expression information (a result of detection; a line coloring) without inhibiting smooth development (flow) of the analyte. The absorption pad section 18, which is provided in order to minimize backflow of the analyte, does not inhibit development (flow) of the analyte even though the absorption pad section 18 abuts against the projected portion 13.

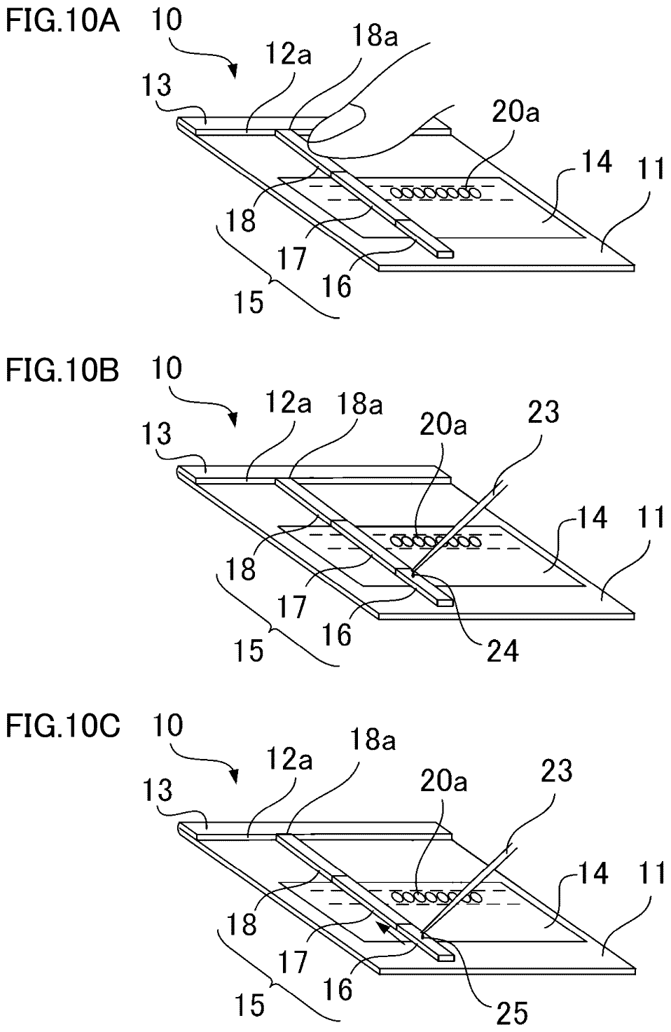

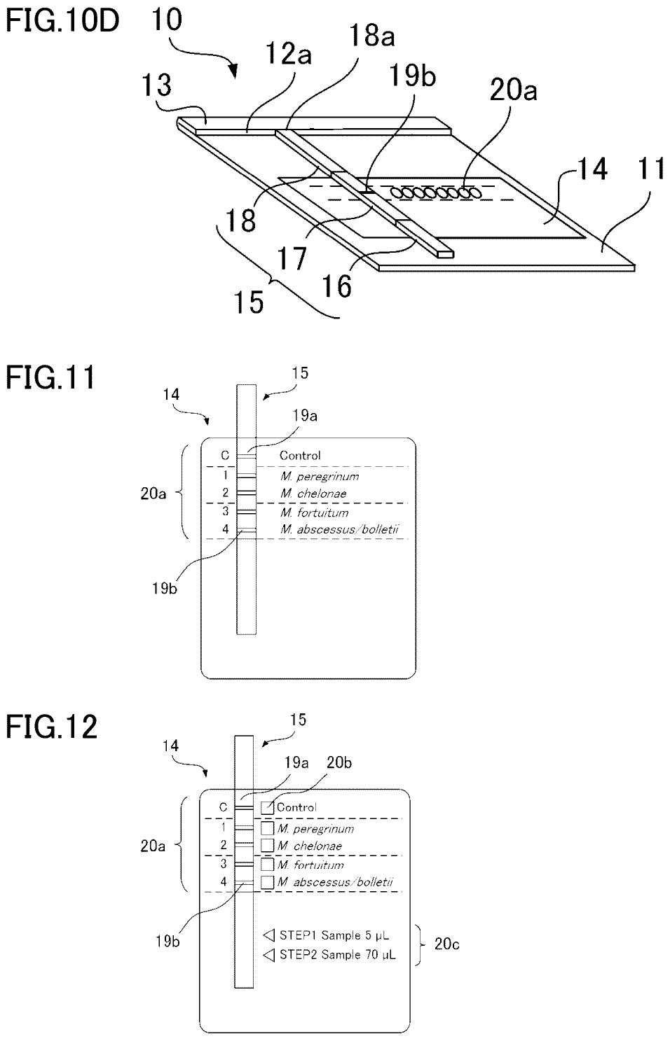

An example of the method of using the chromatographic strip support tool according to the first embodiment will be described with reference to FIG. 10A to FIG. 10D.

As illustrated in FIG. 10A, the chromatographic strip 15 is placed (put) on the support surface 11 of the chromatographic strip support tool 10 such that the extremity 18a of the absorption pad section 18 of the chromatographic strip 15 and the first abutting portion 12a abut against each other.

As illustrated in FIG. 10B, using a sample adding tool 23, a liquid analyte 24 is added onto the analyte supply section 16 of the chromatographic strip 15.

As illustrated in FIG. 10C, using the sample adding tool 23, a developing solution 25 is added onto a location on the analyte supply section 16 of the chromatographic strip 15, the location being closer to the extremity of the analyte supply section 16 than is the location onto which the analyte 24 is added. By the developing solution 25 being added, the analyte 24 and the developing solution 25 are developed through the membrane section 17 in the direction indicated by an arrow in FIG. 10C. Here, the side surfaces of the membrane section 17 do not abut against anything at all. Hence, smooth development of the analyte 24 is not inhibited.

As illustrated in FIG. 10D, when a predetermined biomolecule is contained in the analyte 24, a line coloring 19b occurs at a predetermined location of the membrane section 17 a few minutes after the developing solution 25 is developed. Since the location of the line coloring 19b that has occurred corresponds to the location of the detection item 20a displayed on the display section 14 of the chromatographic strip support tool 10, it is possible to determine that the analyte 24 contains the predetermined biomolecule.

In the first embodiment, the display section 14 illustrated in FIG. 3 is formed on the support surface 11. For example, a display section 14 illustrated in FIG. 11 may be formed on the support surface 11.

The display section 14 illustrated in FIG. 11 displays a plurality of detection items 20a corresponding to a plurality of predetermined locations 19a of the chromatographic strip respectively. Hence, it is possible to obtain plural pieces of expression information (a plurality of results of detection; line colorings) by one testing. In FIG. 11, “M.peregrium”, “M.chelonae”, “M.fortuitum” and “M.abscessus/bolletii” displayed as the detection items represent different kinds of fungi belonging to acid-fast bacilli respectively.

Here, when acquiring result-of-detection image information representing transcribed information regarding the detection items 20a having line colorings 19b that have occurred (expression information) from the first embodiment of the chromatographic strip support tool described with reference to FIG. 2 to FIG. 11, for example, an image of the chromatographic strip support tool may be captured using an image capturing unit after predetermined locations of the detection items 20a corresponding to the line colorings 19b that have occurred (for example, the predetermined locations being the symbols such as “C”, “1”, “2”, and “4” of the detection items 20a in FIG. 5 and FIG. 11) are enclosed with circles. In this way, it is possible to acquire result-of-detection image information.

Other than the example illustrated in FIG. 11, for example, a display section 14 illustrated in FIG. 12 may be formed on the support surface 11. The display section 14 illustrated in FIG. 12 includes the detection items 20a, recording sections 20b, and auxiliary items 20c.

The recording section 20b is not particularly limited and may be appropriately selected in accordance with the intended purpose. Examples of the recording section 20b include a scantron and a checkbox.

In the example illustrated in FIG. 12, for example, when a line coloring 19b occurs on a predetermined location 19a, a person handling the process reads the line coloring 19b and transcribes (records) the reading to the recording section 20b of the detection item 20a. When the person handling the process transcribes (records) the reading of the line coloring 19b to the recording section 20b of the detection item 20a, for example, it is preferable that the person handling the process solidly fills out the recording section 20b.

The location of the recording section 20b is not particularly limited and may be appropriately selected in accordance with the intended purpose. A location adjacent to the detection item 20a is preferable.

The auxiliary item 20c is not particularly limited and may be appropriately selected in accordance with the intended purpose. Examples of the auxiliary item 20c include an indication of the location at which to add an analyte, an indication of the order to add an analyte, and an indication of the amount by which to add an analyte. In FIG. 12, “Δ STEP1 Sample 5 μL” displayed as the auxiliary item represents that “the first thing to do is to add a sample (analyte) of 5 μL to the location indicated by the A symbol (the location being an analyte supply section of a chromatographic strip)”, and “Δ STEP2 Sample 70 μL” displayed as the auxiliary item represents that “the second thing to do is to add a sample (developing solution) of 70 μL to the location indicated by the A symbol (the location being an analyte supply section of a chromatographic strip)”. In this way, the auxiliary item 20c enables even an inexperienced person to use the chromatographic strip support tool.

That is, the example illustrated in FIG. 12 is an example in which a solid fill-out marking by which the person handling the process transcribes a read detection item 20a to a scantron region of the chromatographic strip support tool is used as the result-of-detection image information (an example in which the result-of-detection image information is scantron information). This example can be used as a preferred embodiment of the present invention.

In the first embodiment, paper coated with a water shedding material is used as the material of the support surface 11, the projected portion 13, and the display section 14. In another example, for example, plastics and metals having a water shedding property may be used as the material. By using, for example, plastics and metals having a water shedding property, it is possible to better inhibit soaking with an analyte.

An adhesive label containing analyte information may be pasted on the chromatographic strip support tool. This enables long-term, and associated storage of the analyte information and result-of-detection image information, and enables, for example, collection, storage, and automatic determination in a uniform manner.

The adhesive label is not particularly limited and may be appropriately selected in accordance with the intended purpose. Examples of the adhesive label include a barcode, and a two-dimensional code (QR code (registered trademark)).

The location at which the adhesive label is pasted is not particularly limited and may be appropriately selected in accordance with the intended purpose. Examples of the location include the support surface 11, the projected portion 13, and the display section 14. Among these options, the projected portion 13 is preferable, and a region extending across the projected portion 13 and the one end side of the chromatographic strip support tool 10 in the transverse direction is more preferable. This makes it possible to inhibit detachment of the chromatographic strip 15 from the chromatographic strip support tool 10.

In the first embodiment, the foldable portion 22 is folded along the folding line 21, made to abut against the support surface 11, and fixed. In another example, the projected portion 13 may be laminated and fixed on the support surface 11.

The fixing method is not particularly limited and may be appropriately selected in accordance with the intended purpose so long as two or more plate materials can be joined. Examples of the fixing method include fixing by an adhesive agent.

In the first embodiment, the analyte is supplied to the analyte supply section 16 after the one end side (absorption pad section 18) of the chromatographic strip 15 is placed (put) at the projected portion 13 formed on the support surface 11 of the chromatographic strip support tool 10. In a modification of the first embodiment, the analyte may be supplied to the analyte supply section 16 before the one end side of the chromatographic strip 15 is placed (put) at the projected portion 13 formed on the support surface 11 of the chromatographic strip support tool 10.

### Second Embodiment

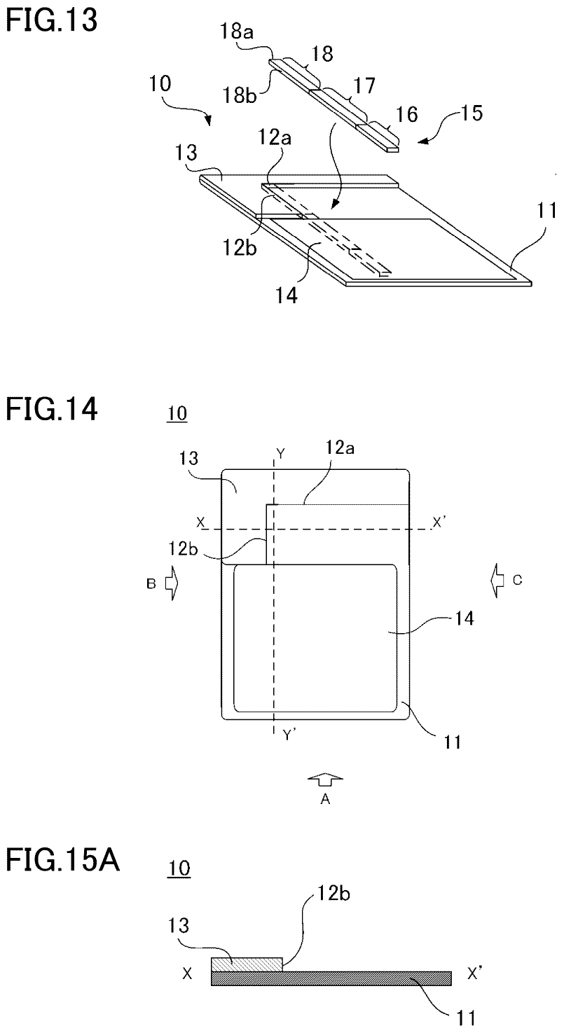

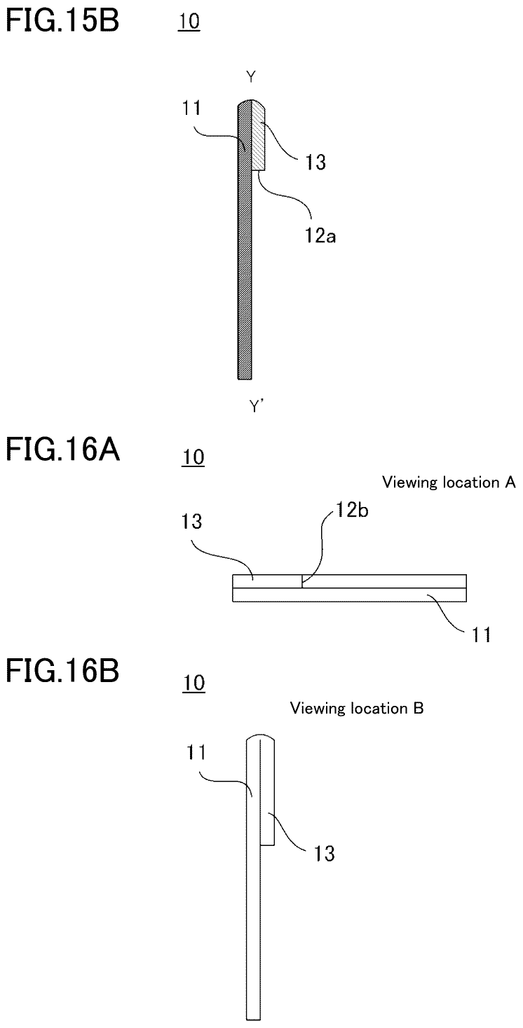

An example of the chromatographic strip support tool according to the second embodiment will be described with reference to FIG. 13 to FIG. 16C.

In the second embodiment, any components that are the same as those included in the modes already described will be denoted by the same reference numerals, and descriptions of such components will be skipped.

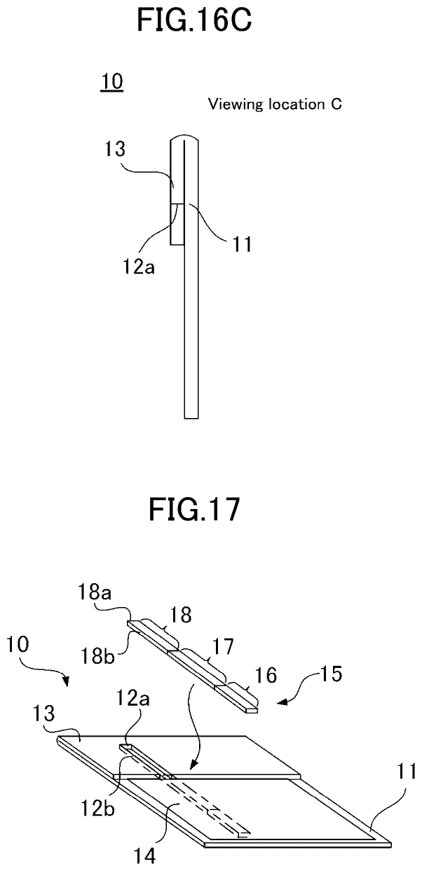

The chromatographic strip support tool 10 according to the second embodiment includes a support surface 11 that is continuous and on which a chromatographic strip 15 can be placed, a projected portion 13 formed on the support surface 11, and a display section 14 formed on the support surface 11 as illustrated in FIG. 13 and FIG. 16C.

The projected portion 13 in the second embodiment has a first abutting portion 12a that can abut against an extremity 18a of the chromatographic strip 15 placed (put) on the support surface 11, the extremity 18a being present at one end side (absorption pad section 18) of the chromatographic strip 15, and a second abutting portion 12b that can abut against at least a partial portion of a side surface 18b of the chromatographic strip 15, the partial portion being present at the one end side (absorption pad section 18) of the chromatographic strip 15, the side surface 18b being parallel with the longitudinal direction of the chromatographic strip 15 as illustrated in FIG. 13 to FIG. 16C.

When placing (putting) the chromatographic strip 15 on the support surface 11, the chromatographic strip 15 is placed (put) on the support surface such that the extremity 18a of the chromatographic strip 15 at the one end side (absorption pad section 18) opposite to the side at which the analyte supply section 16 is present and the first abutting portion 12a abut against each other and at least the partial portion of the side surface 18b of the chromatographic strip 15, the side surface 18b being parallel with the longitudinal direction of the chromatographic strip 15, and the second abutting portion 12b abut against each other. This makes it possible to inhibit mistaken reading of expression information (a result of detection; a line coloring) due to misplacement of the chromatographic strip 15.

As regards the chromatographic strip support tool 10 according to the second embodiment, it is preferable that the first abutting portion 12a and the second abutting portion 12b of the projected portion 13 be disposed at locations at which the first abutting portion 12a and the second abutting portion 12b are orthogonal to each other in a plan view of the support surface 11 as illustrated in FIG. 14. It is also preferable that one end of the first abutting portion 12a and one end of the second abutting portion 12b contact each other in the plan view of the support surface 11.

### Third Embodiment

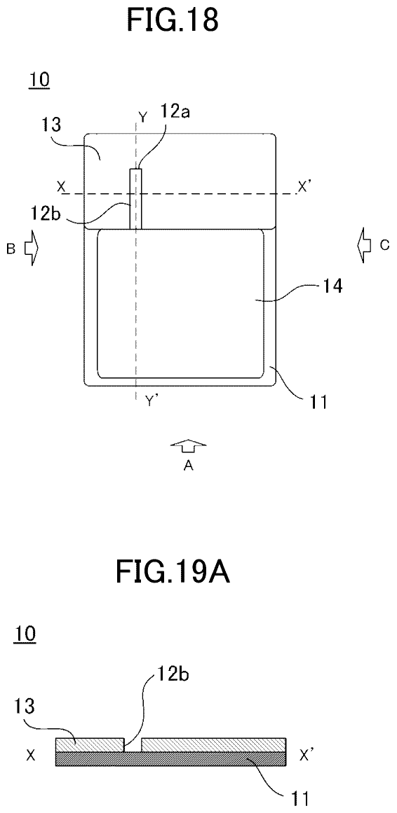

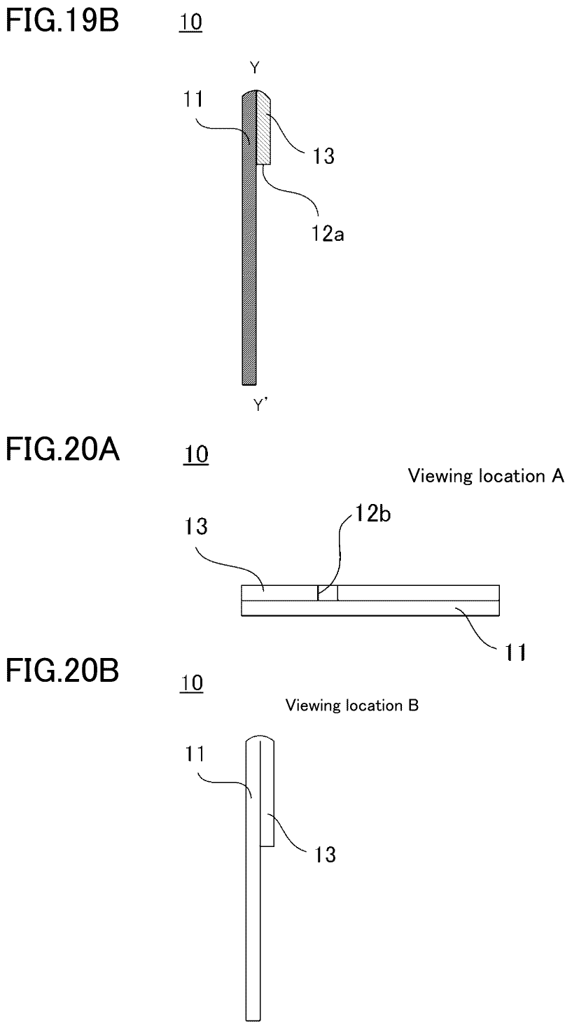

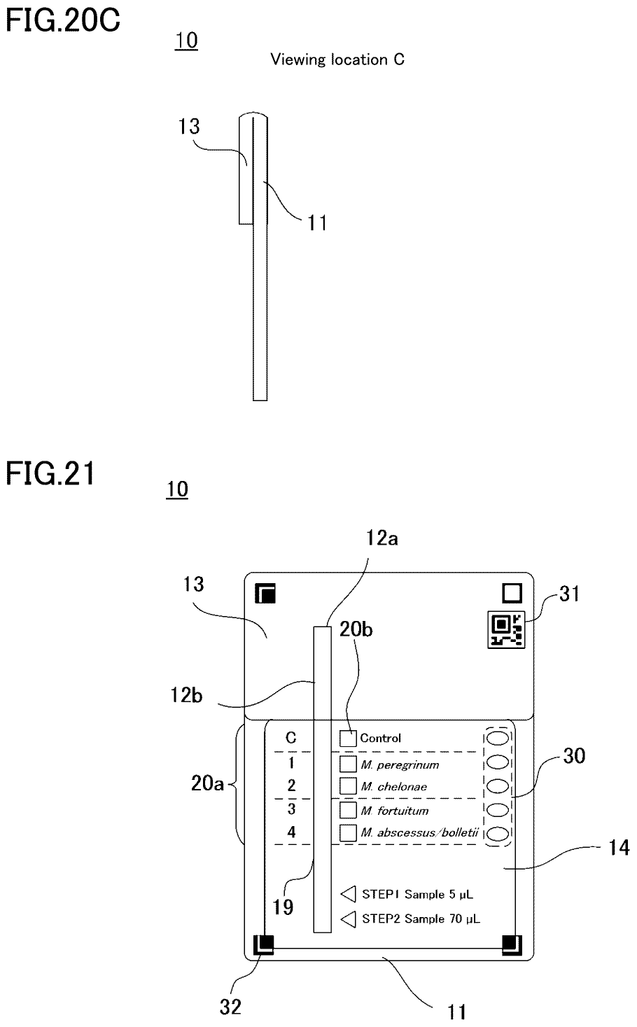

An example of the chromatographic strip support tool according to the third embodiment of the present invention will be described with reference to FIG. 17 to FIG. 22.

In the third embodiment, any components that are the same as those included in the modes already described will be denoted by the same reference numerals, and descriptions of such components will be skipped.

The chromatographic strip support tool 10 according to the third embodiment includes a support surface 11 that is continuous and on which a chromatographic strip 15 can be placed, a projected portion 13 formed on the support surface 11, and a display section 14 formed on the support surface 11 as illustrated in FIG. 17 to FIG. 19C.

The projected portion 13 in the third embodiment has a first abutting portion 12a that can abut against an extremity 18a of the chromatographic strip 15 placed (put) on the support surface 11, the extremity 18a being present at one end side (absorption pad section 18) of the chromatographic strip 15, and a pair of second abutting portions 12b that can abut against at least partial portions of a pair of side surfaces 18b of the chromatographic strip 15 respectively, the partial portions being present at the one end side (absorption pad section 18) of the chromatographic strip 15, the pair of side surfaces 18b being parallel with the longitudinal direction of the chromatographic strip 15 as illustrated in FIG. 17.

When placing (putting) the chromatographic strip 15 on the support surface 11, the chromatographic strip 15 is placed (put) on the support surface such that the extremity 18a of the chromatographic strip 15 at the one end side (absorption pad section 18) opposite to the side at which the analyte supply section 16 is present and the first abutting portion 12a abut against each other and at least the partial portions of the pair of side surfaces 18b of the chromatographic strip 15, the pair of side surfaces 18b being parallel with the longitudinal direction of the chromatographic strip 15, and the pair of second abutting portions 12b abut against each other respectively. This makes it possible to better inhibit mistaken reading of expression information (a result of detection; a line coloring) due to misplacement of the chromatographic strip 15.

FIG. 21 is a plan view illustrating an example of the styles in which various kinds of information carried on a chromatographic strip support tool are displayed according to a third embodiment.

As illustrated in FIG. 21, the chromatographic strip support tool according to the third embodiment includes, for example, an expression information transcribing section 30, a product-in-use identification information indication 31, and an information acquisition assisting marker section 32.

The expression information transcribing section (scantron region) 30 is not particularly limited and may be appropriately selected in accordance with the intended purpose. Examples of the expression information transcribing section include a scantron and a checkbox. For example, a person handling the process may read a line coloring and transcribe (record) the reading to the expression information transcribing section 30. When the person handling the process reads a line coloring and transcribes the reading to the expression information transcribing section 30, it is preferable that, for example, the person handling the process solidly fills out each checkbox of the expression information transcribing section 30. When the person handling the process reads a line coloring and transcribes the reading to the expression information transcribing section 30, for example, the person may transcribe the reading to the expression information transcribing section 30 by using, for example, symbols such as “+” and “−”, or letters.

The product identification information indication 31 is not particularly limited and may be appropriately selected in accordance with the intended purpose. For example, as illustrated in FIG. 21, a two-dimensional code (QR code (registered trademark)) may be used. It is preferable to record, for example, identification information (e.g., an ID) of the chromatographic strip support tool in the product identification information indication 31. For example, the identification information of the chromatographic strip support tool may be selectable from a terminal device owned by a user.

The information acquisition assisting marker section 32 is not particularly limited and may be appropriately selected in accordance with the intended purpose so long as the information acquisition assisting marker section 32 can be used as a marker by which the chromatographic strip support tool is positioned and oriented when acquiring information by capturing a whole image of the chromatographic strip support tool using an information acquiring unit such as an image capturing unit. It is preferable to provide the information acquisition assisting marker section 32 at the four corners of the chromatographic strip support tool when the planar shape of the chromatographic strip support tool is a quadrangular shape as in the example illustrated in FIG. 21.

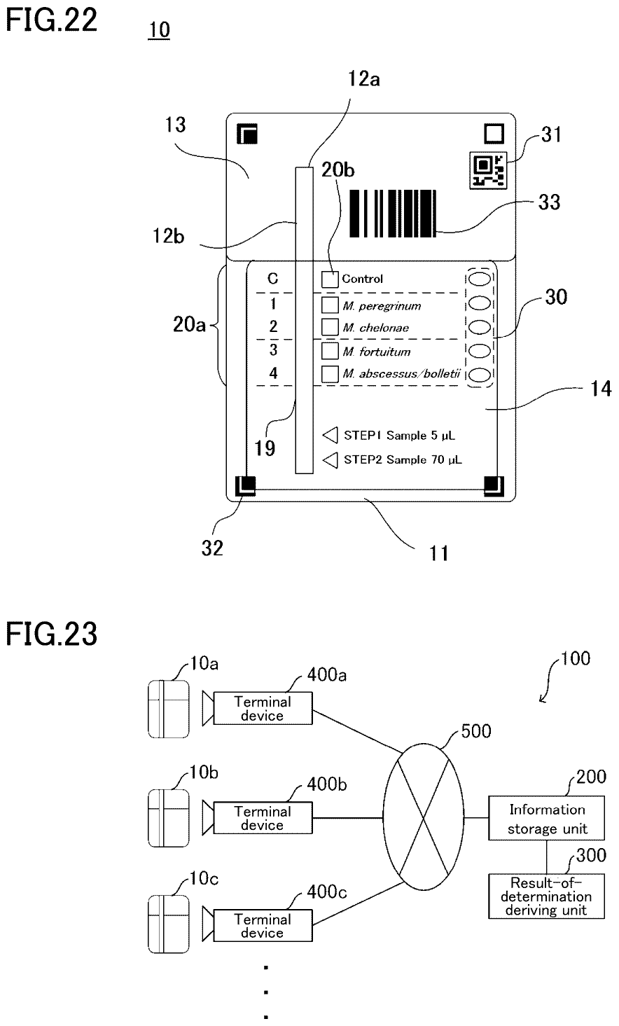

FIG. 22 is a plan view illustrating another example of the styles in which various kinds of information carried on the chromatographic strip support tool are displayed according to the third embodiment.

Unlike the example illustrated in FIG. 21, the chromatographic strip support tool according to the third embodiment illustrated in FIG. 22 further includes an analyte information indication 33.

The analyte information indication 33 is not particularly limited and may be appropriately selected in accordance with the intended purpose. For example, as illustrated in FIG. 22, a barcode may be used. As the analyte information, for example, information indicating the subject who has donated the analyte, information indicating the date on which the analyte is collected, and information regarding, for example, the hospital at which the analyte is collected may be recorded in the analyte information indication 33.

In a preferable mode, for example, the subject (patient) itself, who has donated the analyte, or the tester (e.g., a nurse) applies the analyte information indication 33 on the chromatographic strip support tool. In this case, it is preferable to use an adhesive seal on which a barcode is printed, as the analyte information indication 33.

When acquiring information by capturing a whole image of the chromatographic strip support tool illustrated in FIG. 22 using an information acquiring unit such as an image capturing unit, and storing the acquired information in the information storage unit, for example, it is optional whether to select and store any of the result-of-detection transcribing section 30, the product-in-use identification information indication 31, and the analyte information indication 33, or to store the whole image of the chromatographic strip support tool as is.

### <Embodiments of Automatic Determination System>

The embodiments of the automatic determination system of the present invention will be described below with reference to the drawings.

FIG. 23 is a block diagram illustrating an example of a configuration of the automatic determination system according to an embodiment of the present invention.

As illustrated in FIG. 23, the automatic determination system 100 includes an information storage unit 200, a result-of-determination deriving unit 300, and terminal devices 400a, 400b, 400c, . . . . In the automatic determination system 100, the information storage unit 200 and the result-of-determination deriving unit 300 are communicably connected. In the automatic determination system 100, the terminal devices 400a, 400b, 400c, . . . are each communicably connected to the information storage unit 200 via a network 500.

The terminal devices 400a, 400b, 400c, . . . each include an image capturing unit, which is an example of the information acquiring unit, and can acquire information from chromatographic strip support tools 10a, 10b, 10c . . . .

The terminal devices 400a, 400b, 400c, . . . have the same device configuration. Hence, they will be referred to as “terminal device 400” and described collectively.

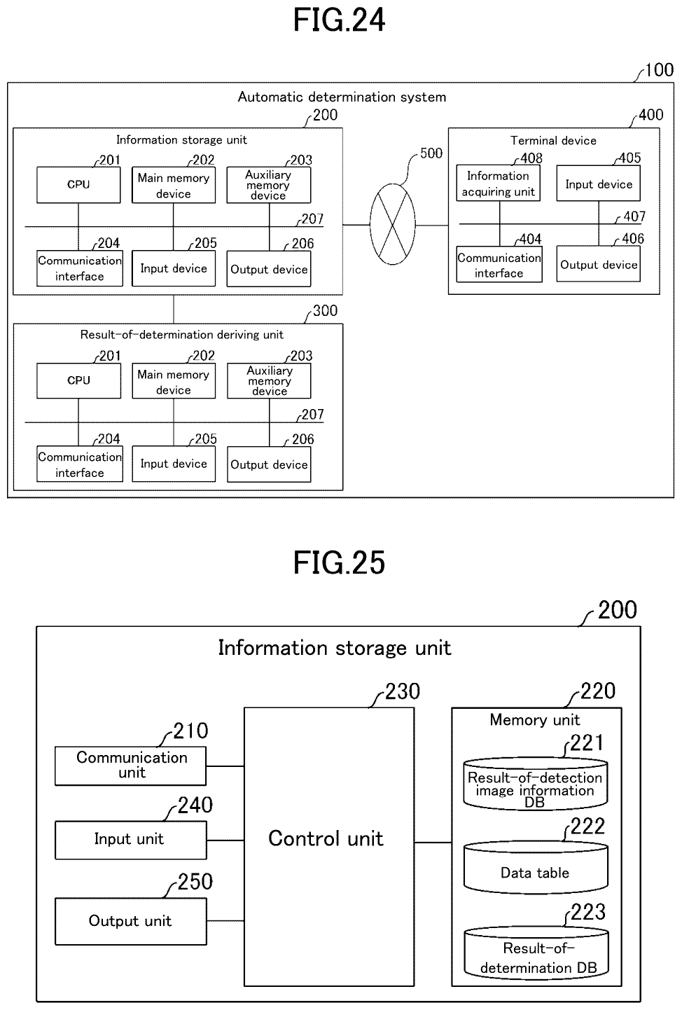

FIG. 24 is a block diagram illustrating an example of the hardware configuration of the automatic determination system according to an embodiment of the present invention.

As illustrated in FIG. 24, the information storage unit 200 of the automatic determination system 100 includes a Central Processing Unit (CPU) 201, a main memory device 202, an auxiliary memory device 203, a communication interface 204, an input device 205, and an output device 206. These components are mutually connected via a bus 207.

The CPU 201 is a processing device configured to perform various controls and operations. The CPU 201 realizes various functions by executing, for example, programs read into, for example, the main memory device 202. That is, the CPU 201 executes various programs (e.g., the automatic determination program) of the automatic determination system 100.

The CPU 201 can control the whole operations of the information storage unit 200 of the automatic determination system 100. In the present embodiment, the device configured to control the whole operations of the information storage unit 200 of the automatic determination system 100 is the CPU 201. However, the device is not limited to the CPU 201, but may be, for example, a Field Programmable Gate Array (FPGA).

The main memory device 202 is configured to store various programs and to also store data necessary for execution of the various programs.

For example, the main memory device 202 includes either or both of a ROM and a Random Access Memory (RAM).

The ROM stores various programs such as, for example, a Basic Input/Output System (BIOS), and the automatic determination program of the present invention. The ROM is not particularly limited and may be appropriately selected in accordance with the intended purpose. Examples of the ROM include a masked ROM, and a Programmable ROM (PROM).

The RAM functions as a work area in which various programs stored in, for example, the ROM and the auxiliary memory device are unfolded when they are executed by the CPU 201. The RAM is not particularly limited and may be appropriately selected in accordance with the intended purpose. Examples of RAM include a Dynamic Random Access Memory (DRAIM) and a Static Random Access Memory (SRAM).

The auxiliary memory device 203 is not particularly limited and may be appropriately selected in accordance with the intended purpose so long as various kinds of information can be stored. Examples of the auxiliary memory device include a Solid State Drive (SSD), and a hard disk drive (HDD). The auxiliary memory device 203 may be a portable memory device such as a CD drive, a DVD drive, and a Blu-ray (registered trademark) Disc (BD) drive.

The communication interface 204 is not particularly limited, and a publicly-known communication interface may be appropriately used. Examples of the communication interface include wireless (e.g., Bluetooth (registered trademark) and Wi-Fi (registered trademark)) communication devices, and wired communication devices. The information storage unit 200 collects, for example, result-of-detection image information by communicating with a terminal (terminal device 400) owned by a user via a network 500 by means of the communication interface 204.

The input device 205 is not particularly limited so long as the input device can receive inputs of various requests and information into the information storage unit 200. A publicly-known input device may be appropriately used. Examples of the input device include a keyboard, a mouse, a touch panel, and a mike. When the input device 205 is a touch panel (touch display), the input device 205 can double-function as the output device 206.

The output device 206 is not particularly limited, and a publicly-known output device may be appropriately used. Examples of the output device include a display. Examples of the display used as the output device 206 include a liquid crystal display and an organic Electro Luminescence (EL) display.

The result-of-determination deriving unit 300 of the automatic determination system 100 includes a CPU 301, a main memory device 302, an auxiliary memory device 303, a communication interface 304, an input device 305, and an output device 306. These components are mutually connected via a bus 307.

As each of the components of the result-of-determination deriving unit 300, the same one as that of the information storage unit 200 described above can be used.

The terminal device 400 of the automatic determination system 100 includes at least a communication interface 404, an input device 405, an output device 406, and an information acquiring unit 408. These components are mutually connected via a bus 407. As the terminal device 400, for example, a smartphone, a tablet terminal, and a personal computer (PC) can be used.

As the information acquiring unit 408, for example, an image capturing unit (e.g., a camera) can be used.

The details of the terminal device will be described below.

The automatic determination system 100 may be a part of a cloud, which is a group of computers on a network.

FIG. 25 is a block diagram illustrating an example of the functional configuration of the information storage unit of the automatic determination system according to an embodiment of the present invention.

As illustrated in FIG. 25, the information storage unit 200 of the automatic determination system 100 includes a communication unit 210, a memory unit 220, a control unit 230, an input unit 240, and an output unit 250.

In the information storage unit 200, for example, the function of the communication unit 210 is realized by the communication interface 204, the function of the memory unit 220 is realized by the main memory device 202 and the auxiliary memory device 203, the function of the control unit 230 is realized by the CPU 201 and the main memory device 202, the function of the input unit 240 is realized by the input device 205, and the function of the output unit 250 is realized by the output device 206.

For example, the communication unit 210 is configured to exchange various data with an external device (e.g., the terminal device 400). For example, the information storage unit 200 is configured to acquire, for example, result-of-detection image information by communicating with a terminal device 400 via the network 500 by means of the communication unit 210.

For example, the memory unit 220 stores various programs and data. The memory unit 220 includes a result-of-detection image information database (DB) 221, a data table 221, and a result-of-determination database (DB) 222.

The result-of-detection image information DB 221 is not particularly limited and may be appropriately selected in accordance with the intended purpose so long as it is a database that can store result-of-detection image information acquired by a terminal device 400.

The data table 222 is not particularly limited and may be appropriately selected in accordance with the intended purpose so long as image information including transcribed information regarding expression information and a result of determination (result of testing) on a chromatographic strip are associated with each other in the data table. For example, a data table as illustrated in FIG. 2 may be used.

The result-of-determination DB 223 is not particularly limited and may be appropriately selected in accordance with the intended purpose so long as it can store a result of determination derived by the result-of-determination deriving unit 300 in association with analyte information and result-of-detection image information. For example, a DB including a result-of-determination table as illustrated in FIG. 3 may be used.

In the present embodiment, it is optional to realize the information storage unit 300 by using an area on a cloud server.

For example, the control unit 230 is configured to execute the various programs stored in the memory unit 220 and control the whole operations of the information storage unit 200.

The input unit 240 is configured to receive various instructions to the information storage unit 200.

For example, the output unit 250 is configured to output various information for view.

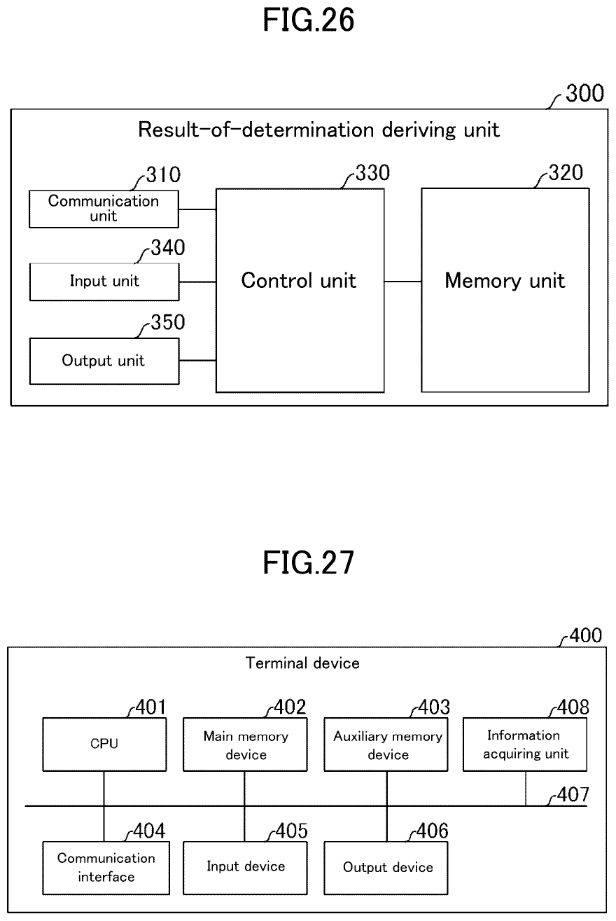

FIG. 26 is a block diagram illustrating an example of the functional configuration of the result-of-determination deriving unit of the automatic determination system according to an embodiment of the present invention.

As illustrated in FIG. 26, the result-of-determination deriving unit 300 includes a communication unit 310, a memory unit 320, a control unit 330, an input unit 340, and an output unit 350.

The communication unit 310, the control unit 330, the input unit 340, and the output unit 350 of the result-of-determination deriving unit 300 can be the same as the communication unit 210, the control unit 230, the input unit 240, and the output unit 250 of the information storage unit 200.

For example, using the data in the result-of-detection image information DB 221 and the data table 222 stored in the information storage unit 200, the result-of-determination deriving unit 300 checks an image included in result-of-detection image information against the data table 222, derives a result of determination included in the data table 222 and corresponding to the image, and records (stores) the result of determination in the result-of-determination DB 223.

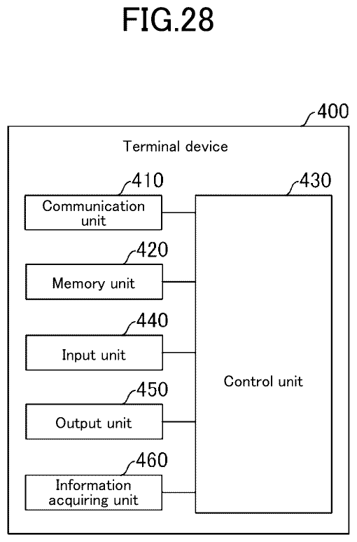

FIG. 27 is a block diagram illustrating an example of the hardware configuration of the terminal device of the automatic determination system according to an embodiment of the present invention. FIG. 28 is a block diagram illustrating an example of the functional configuration of the terminal device of the automatic determination system according to an embodiment of the present invention.

The terminal device 400 illustrated in FIG. 24 will be described in greater detail with reference to FIG. 27 and FIG. 28.

As illustrated in FIG. 27, the terminal device 400 includes a CPU 401, a main memory device 402, an auxiliary memory device 403, a communication interface 404, an input device 405, an output device 406, and an information acquiring unit 408. These components are mutually connected via a bus 407.

The CPU 401, the main memory device 402, the auxiliary memory device 403, the communication interface 404, the input device 405, and the output device 406 of the terminal device 400 may be the same as the CPU 201, the main memory device 202, the auxiliary memory device 203, the communication interface 204, the input device 205, and the output device 206 of the information storage unit 200.

The terminal device 400 can send, for example, result-of-detection image information to the information storage unit 200 by communicating with the information storage unit 200 via the network 500 by means of the communication interface 404.

The information acquiring unit 408 of the terminal device 400 may be, for example, an image capturing unit such as a camera. The information acquiring unit 408 of the terminal device 400 can capture an image of the chromatographic strip support tool 10 and acquire information such as result-of-detection image information.

As illustrated in FIG. 28, the terminal device 400 of the automatic determination system 100 includes a communication unit 410, a memory unit 420, a control unit 430, an input unit 440, an output unit 450, and an information acquiring unit 460.

The communication unit 410, the memory unit 420, the control unit 430, the input unit 440, and the output unit 450 of the terminal device 400 may be the same as the communication unit 210, the memory unit 220, the control unit 230, the input unit 240, and the output unit 250 of the information storage unit 200.

The information acquiring unit 460 of the terminal device 400 captures an image of the chromatographic strip support tool 10 and acquires information such as result-of-detection image information. The function of the information acquiring unit 460 is realized by the information acquiring unit 408.

In FIG. 23 to FIG. 28, a mode in which the information storage unit 200 and the result-of-determination deriving unit 300 are provided independently has been described. However, the present invention is not limited to this mode. For example, a mode in which the information storage unit 200 and the result-of-determination deriving unit 300 are integrated (for example, realized by one server device) is possible.

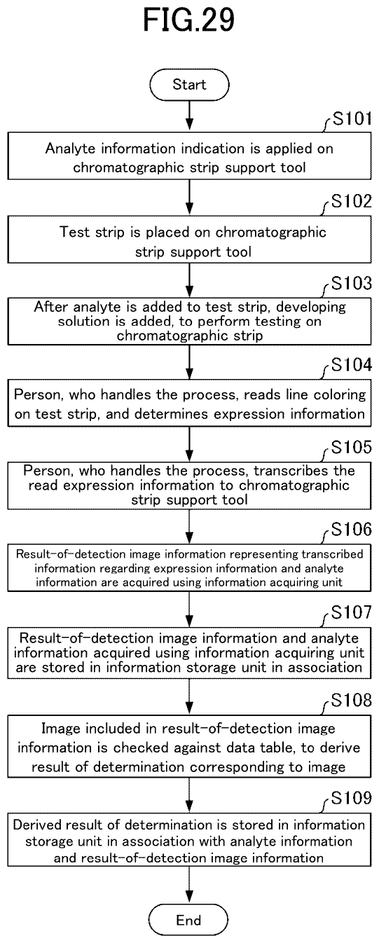

FIG. 29 is a flowchart illustrating an example of the flow of automatic determination on a chromatographic strip that has been through testing, using the automatic determination system of the present invention. Here, the flow of automatic determination on a chromatographic strip that has been through testing, using the automatic determination system of the present invention will be described for each step denoted by S in the flowchart illustrated in FIG. 29.

In the step S101, an analyte information indication is applied on the chromatographic strip support tool. More specifically, in the step S101, for example, the subject who has donated the analyte pastes an adhesive seal, on which analyte information including information regarding the subject's own analyte is printed in the form of a barcode, on the chromatographic strip support tool.

Next, in the step S102, a test strip is placed on the chromatographic strip support tool. More specifically, for example, the person handling the process and performing the testing places a test strip on a predetermined location of the chromatographic strip support tool.

Next, in the step S103, after the analyte is added to the test strip, a developing solution is added, to perform testing on the chromatographic strip. More specifically, in the step S103, for example, the person handling the process and performing the testing supplies the analyte to the test strip and then adds a developing solution, to develop the analyte at a predetermined location of the test strip and perform testing by the chromatographic strip. Here, an example in which testing by the chromatographic strip is performed after the test strip is placed on the chromatographic strip support tool has been illustrated. However, for example, after the analyte is developed at a predetermined location of the test strip and testing by the chromatographic strip is performed, the chromatographic strip that has been through testing may be placed on the chromatographic strip support tool.

Next, in the step S104, the person handling the process reads a line coloring on the test strip, and determines the expression information. More specifically, in the step S104, for example, when a line coloring occurs on a predetermined location of the test strip, the person handling the process reads the line coloring (visually), determines the detection item corresponding to the location of the line coloring, and determines the expression information.

Then, in the step S105, the person handling the process transcribes the read expression information to the chromatographic strip support tool. More specifically, in the step S105, for example, the person handling the process and performing the testing transcribes the expression information determined by reading the line coloring in the step S104 to the scantron region of the chromatographic strip support tool. In the step S105, for example, it is preferable that the person handling the process transcribes the expression information by solidly filling out a checkbox in the scantron region.

Next, in the step S106, result-of-detection image information representing the transcribed information regarding the expression information and the analyte information are acquired using the information acquiring unit. More specifically, in the step S106, for example, an image of the chromatographic strip support tool is captured using the image capturing unit serving as the information acquiring unit, to acquire result-of-detection image information and the analyte information.

Next, in the step S107, the result-of-detection image information and the analyte information acquired using the information acquiring unit are stored in the information storage unit in association. More specifically, in the step 3107, for example, the result-of-detection image information and the analyte information are sent from the information acquiring unit to the information storage unit and stored in the data table, in which result-of-detection image information and analyte information are associated, in the information storage unit.

Next, in the step S108, an image included in the result-of-detection image information is checked against the data table, to derive a result of determination corresponding to the image. More specifically, in the step S108, for example, using the result-of-detection image information and the data in the data table stored in the information storage unit, an image included in the result-of-detection image information is checked against the data table, and a result of determination included in the data table and corresponding to the image is derived.

Then, in the step S109, the derived result of determination is stored in association with the analyte information and the result-of-detection image information, and the process is terminated. More specifically, in the step S109, for example, the result of determination derived in the step S108 is stored in the data table, in which result-of-detection image information and analyte information are associated, in the information storage unit, and the process is terminated.

Here, the steps being performed in the specific order according to the flowchart illustrated in FIG. 29 has been described. However, the present invention is not limited to performing the steps in the order of the flowchart illustrated in FIG. 29. So long as technical inconsistency will not occur, the order may be changed appropriately, and a plurality of steps may be performed simultaneously.

As described above, the automatic determination system of the present invention includes an information storage unit configured to store result-of-detection image information acquired from a chromatographic strip support tool carrying the result-of-detection image information, the result-of-detection image information representing transcribed information regarding expression information obtained on a chromatographic strip that has been through testing, and a result-of-determination deriving unit configured to check an image included in the result-of-detection image information against a data table and derive a result of determination included in the data table and corresponding to the image.

In this way, the automatic determination system of the present invention can store information that enables discerning of expression information on a chromatographic strip that has been through testing, in the form of uniform information that can be stored for a long term, and can accurately derive a result of determination on the chromatographic strip based on the information that enables discerning of the expression information.

### REFERENCE SIGNS LIST

10: chromatographic strip support tool
11: support surface
12a: first abutting portion
12b: second abutting portion
13: projected portion
14: display section
15: chromatographic strip
16: analyte supply section
17: membrane section
18: absorption pad section
18a: extremity
18b: side surface
19a: predetermined location
19b: line coloring
20a: detection item
20b: recording section
20c: auxiliary item
21: folding line
22: foldable portion
30: expression information transcribing section (scantron region)
31: product-in-use identification information indication
32: information acquisition assisting marker section
33: analyte information indication
100: automatic determination system
200: information storage unit
300: result-of-determination deriving unit
400: terminal device
500: network

1. An automatic determination system, comprising:
an information storage unit configured to store result-of-detection image information acquired from a chromatographic strip support tool carrying the result-of-detection image information, the result-of-detection image information representing transcribed information regarding expression information obtained on a chromatographic strip that has been through testing; and
a result-of-determination deriving unit configured to check an image included in the result-of-detection image information against a data table, and derive a result of determination included in the data table and corresponding to the image.

2. The automatic determination system according to claim 1,
wherein the information storage unit is configured to store analyte information in association with the result-of-detection image information, the analyte information being acquired from the chromatographic strip support tool further carrying the analyte information, the analyte information being regarding an analyte supplied to the chromatographic strip.

3. The automatic determination system according to claim 2,
wherein the information storage unit is configured to store the result of determination derived by the result-of-determination deriving unit in association with the analyte information and the result-of-detection image information.

4. The automatic determination system according to claim 2,
wherein the analyte information is code information, and the result-of-detection image information is scantron information.

5. The automatic determination system according to claim 2, further comprising:
an information acquiring unit configured to acquire either or both of the analyte information and the result-of-detection image information.

6. The automatic determination system according to claim 5,
wherein the information acquiring unit is an image capturing unit.

7. An automatic determination method, comprising:
storing result-of-detection image information acquired from a chromatographic strip support tool carrying the result-of-detection image information, the result-of-detection image information representing transcribed information regarding expression information obtained on a chromatographic strip that has been through testing; and
checking an image included in the result-of-detection image information against a data table, and deriving a result of determination included in the data table and corresponding to the image.

8. A computer-readable recording medium having stored therein an automatic determination program causing a computer to execute:
storing result-of-detection image information acquired from a chromatographic strip support tool carrying the result-of-detection image information, the result-of-detection image information representing transcribed information regarding expression information obtained on a chromatographic strip that has been through testing; and
checking an image included in the result-of-detection image information against a data table, and deriving a result of determination included in the data table and corresponding to the image.
